(19) United States
(12) Patent Application Publication
(10) Pub. No.: US 2024/0425573 A1
(43) Pub. Date: Dec. 26, 2024

(54) METHODS FOR TREATING ALZHEIMER'S DISEASE

(71) Applicant: Biogen MA Inc., Cambridge, MA (US)

(72) Inventors: Laura Nisenbaum, Cambridge, MA (US); Tianle Chen, Newton, MA (US); Raj Rajagovindan, Winchester, MA (US)

(21) Appl. No.: 18/707,366

(22) PCT Filed: Nov. 2, 2022
(86) PCT No.: PCT/US22/48684

§ 371 Date: May 3, 2024

Related U.S. Application Data
(60) Provisional application No. 63/343,029, filed on May 17, 2022.
(60) Provisional application No. 63/309,796, filed on Feb. 14, 2022.
(60) Provisional application No. 63/277,127, filed on Nov. 8, 2021.
(60) Provisional application No. 63/275,442, filed on Nov. 3, 2021.

(51) Int. Cl. C07K 16/18; A61K 39/00; A61P 25/28; G01N 33/68

(52) U.S. Cl. CPC C07K 16/18; A61P 25/28; G01N 33/6896; A61K 2039/505; A61K 2039/55; C07K 2317/565; G01N 2333/4709; G01N 2800/52

(57) ABSTRACT

Provided are methods for treating Alzheimer's disease in a human subject in need thereof comprising administration of an anti-beta-amyloid antibody (e.g., aducanumab) to the subject.

### FIELD OF THE INVENTION

This disclosure relates generally to methods for treating Alzheimer's disease.

### BACKGROUND

Alzheimer's disease (AD) is a progressive neurodegenerative disorder clinically characterized by cognitive impairment, behavioral disturbances, psychiatric symptoms, and disability in activities of daily living. These clinical manifestations constitute AD dementia.

AD International estimates that the number of people living with dementia worldwide will increase from the current value of 47 million to 131 million by 2050. Being the most common cause of dementia, AD accounts for 60 to 80% of dementia cases. In the United States, it is estimated that 5.2 million Americans suffer from dementia caused by AD, and that by 2050 the prevalence will double or triple unless an effective treatment is found.

Thus, there exists an ongoing need for methods to treat Alzheimer's disease patients.

### SUMMARY

The current disclosure describes, among other things, methods of treating Alzheimer's disease that include administering an anti-beta-amyloid antibody. The present disclosure encompasses a recognition that there is a correlation between, e.g., plasma p-tau levels, amyloid beta plaque levels, and clinical progression of Alzheimer's disease in subjects being treated with an anti-beta-amyloid antibody, e.g., aducanumab. Accordingly, the present disclosure provides the insight that a p-tau level in plasma of a human subject can be used to assess and/or adjust treatment with an anti-beta-amyloid antibody.

Moreover, the present disclosure provides the recognition that plasma p-tau levels can be useful identifying subgroups that may experience improved benefits from treatment with an anti-beta-amyloid antibody, e.g., aducanumab. Accordingly, the present disclosure provides the insight that plasma p-tau levels can be useful in identifying and/or selecting patients for treatment with an anti-beta-amyloid antibody, e.g., aducanumab. The current disclosure also provides that plasma p-tau levels can be useful for monitoring a subject receiving a treatment with an anti-beta-amyloid antibody, e.g., aducanumab, and/or determining whether such a subject should continue to receive a treatment including an anti-beta-amyloid antibody, e.g., aducanumab.

Plasma p-tau measurements can provide benefits over present techniques, which can be invasive, painful, and time consuming. Allowing subjects (e.g., patients) and physicians access to information about how a subject is responding to treatment with an anti-beta-amyloid antibody, e.g., aducanumab, the technologies described herein can improve subject outcomes and increase subject compliance for a subject population that is already experiencing challenging signs and symptoms, and may be prone to lapses in treatment.

Moreover, the present disclosure provides the insight the that plasma p-tau levels in subjects show a dose-dependent and time-dependent correlation with aducanumab treatment. These correlations provide subjects and physicians with a meaningful way to determine whether administration of aducanumab is effective for treatment of Alzheimer's Disease in a subject. For example, plasma p-tau levels in the plasma of a subject may be useful in determining an aducanumab treatment regiment that results in a subject's Alzheimer's Disease being treated, while minimizing adverse events (e.g., ARIA).

Additionally, the present disclosure evidences that plasma p-tau levels may be sufficient for conducting certain assessments and/or monitoring. As such, in some embodiments described herein, methods do not comprise assessments and/or monitoring of other biomarkers, e.g., methods described herein may not comprise assessments and/or monitoring of CSF p-tau levels, amyloid plaques, or αβ42/40 ratios.

In some embodiments, the present disclosure provides evidence suggesting that a baseline level of plasma p-tau may correlate with aducanumab treatment response. The present disclosure demonstrates that patients with a higher plasma p-tau level at baseline (e.g., those in the fourth quartile of baseline plasma p-tau levels) show improved response to aducanumab treatment. The present disclosure encompasses a recognition that a baseline level of plasma p-tau may be useful in determining whether administration of aducanumab is effective for treatment of Alzheimer's Disease in a subject. Thus, in some embodiments, a baseline level of plasma p-tau may be used to select a patient or patient population for treatment with aducanumab. In some embodiments, methods described herein can comprise measuring a baseline level of plasma p-tau.

In some embodiments, the present disclosure provides methods comprising (i) determining a p-tau level in plasma of a human subject, and (ii) administering one or more doses of an anti-beta-amyloid antibody to the human subject.

In some embodiments, a p-tau level in plasma of a human subject is determined prior to the human subject receiving a dose of the anti-beta-amyloid antibody. In some embodiments, provided methods further comprise determining a p-tau level in plasma of a human subject at a first and second time point. In some embodiments, a first time point for determining a p-tau level is prior to the human subject receiving a dose of the anti-beta-amyloid antibody. In some embodiments, a second time point for determining a p-tau level is after the human subject has received at least one dose of the anti-beta-amyloid antibody. In some embodiments, a second time point for determining the level of plasma p-tau is after at least 10 doses of anti-beta-amyloid antibody have been administered to the human subject. In some embodiments, a second time point for determining the level of plasma p-tau is after at least 14 doses of anti-beta-amyloid antibody have been administered to the human subject. In some embodiments, a second time point for determining the level of plasma p-tau is after at least 19 doses of anti-beta-amyloid antibody have been administered to the human subject.

In some embodiments, the human subject is administered an anti-beta-amyloid antibody periodically, e.g., every 4 weeks, over a period of 56 weeks, 78 weeks, 104 weeks, 128 weeks, 134 weeks, or longer.

In some embodiments, a p-tau level in plasma of a human subject is determined at a first and second time point, where the first time point occurs before the second time point, and, if the level of plasma p-tau at a first time point is greater than the level of plasma p-tau at the second time point, the method further comprises a second or subsequent administration of the anti-beta-amyloid antibody to the human subject. In some embodiments, the second or subsequent administration of the anti-beta-amyloid antibody includes a dose of the anti-beta-amyloid antibody that is equal to or greater than the dose of the anti-beta-amyloid antibody administered to the human subject in the first administration.

In some embodiments, a p-tau level in plasma of a human subject is determined at a first and second time point, where the first time point occurs before the second time point, and, if the level of plasma p-tau at a first time point is less than the level of plasma p-tau at the second time point, the method further comprises a second or subsequent administration of the anti-beta-amyloid antibody to the human subject. In some embodiments, the second or subsequent administration of the anti-beta-amyloid antibody includes a dose of the anti-beta-amyloid antibody that is equal to or less than the dose of the anti-beta-amyloid antibody administered to the human subject in the first administration.

In some embodiments, a p-tau level in plasma of a human subject is determined at a first and second time point, where the first time point is a baseline or reference measurement, and the second time point occurs after administering one or more doses of anti-beta-amyloid antibody. In some embodiments, the first time point is a baseline at time point that occurs before administering anti-beta-amyloid antibody. In some embodiments, a p-tau level in plasma at a first time point is a baseline that is adjusted. In some embodiments, a p-tau level in plasma at a first time point is a baseline that is unadjusted. In some embodiments, a p-tau level in plasma at a second time point is reduced at least 5%, at least 6%, at least 7%, at least 8%, at least 9%, at least 10%, at least 11%, at least 12%, at least 13%, at least 14%, or at least 15% relative to the first time point (e.g., baseline). In some embodiments, a p-tau level in plasma at a second time point is reduced at least 10% relative to the first time point (e.g., baseline).

In some embodiments, a p-tau level in plasma of a human subject is compared to a reference measurement of p-tau in plasma. In some embodiments, a reference measurement of p-tau in plasma is, e.g., from healthy human subjects or human subjects diagnosed with AD that have not received treatment.

In some embodiments, a p-tau level in plasma of a human subject is compared to a reference level of p-tau of subjects who have been administered placebo. In some embodiments, a p-tau level in plasma of a human subject is reduced least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 24%, or at least 25% relative to p-tau levels of a placebo group of patients at the same time point.

In some embodiments, a p-tau level in plasma of a human subject has an adjusted mean change from baseline that is reduced relative to the adjusted mean change from baseline of a placebo group of patients at the same time point. In some embodiments, a p-tau level in plasma of a human subject has an adjusted mean change from baseline that is reduced at least −0.2, −0.25, −0.3, −0.35, −0.4, −0.45, −0.5, −0.55, −0.6, −0.65, −0.7, −0.75, −0.8, −0.85, −0.9, −0.95, relative to the adjusted mean change from baseline of a placebo group of patients at the same time point. In some embodiments, the time point is after a period of 56 weeks, 78 weeks, 104 weeks, 128 weeks, 134 weeks, or longer.

In some embodiments, after 56 weeks of administering, a p-tau level in plasma of a human subject has an adjusted mean change from baseline that is reduced within a range of −0.1 to −0.6 relative to the adjusted mean change from baseline of a placebo group of patients at the same time point.

In some embodiments, after 78 weeks of administering, a p-tau level in plasma of a human subject has an adjusted mean change from baseline that is reduced within a range of −0.2 to −0.7 relative to the adjusted mean change from baseline of a placebo group of patients at the same time point.

In some embodiments, the present disclosure provides methods comprising (i) determining a p-tau level in plasma of a human subject at baseline, and to a subjects whose plasma p-tau level is above a reference level (ii) administering one or more doses of an anti-beta-amyloid antibody.

In some embodiments, a p-tau level in plasma comprises tau with phosphorylation at one or more sites selected from: Y18, S46, T50, S69, T71, S113, T123, T153, T175, T181, S184, S85, S191, Y197, S198, S199, S202, T205, S208, S210, T212, S214, T217, T231, S235, S237, S238, S258, S262, S289, S356, Y397, S400, T403, S404, S409, S412, S413, T414/S416, S422, T427, S433, and S435. In some embodiments, a p-tau level in plasma comprises p-tau¹⁸¹ and/or p-tau²¹⁷. In some embodiments, the p-tau level is a plasma p-tau¹⁸¹ level.

In some embodiments, a human subject has or is suspected of having a disease associated with the accumulation and deposition of beta amyloid, such as Alzheimer's disease, Down's syndrome, mild cognitive impairment, cerebral amyloid angiopathy, vascular dementia, and/or multi-infarct dementia.

In some embodiments, a human subject has been diagnosed with mild cognitive impairment due to Alzheimer's disease. In some embodiments, a subject has been diagnosed with mild cognitive impairment due to Alzheimer's disease-intermediate likelihood by a CDR global score of 0.5 and a Memory Box score of 0.5 or greater before treatment. In some embodiments, a subject has been diagnosed with mild cognitive impairment due to Alzheimer's disease-intermediate likelihood by a history of subjective memory decline with gradual onset and slow progression over the last 1 year before treatment, e.g., as corroborated by an caregiver. In some embodiments, a subject has been diagnosed with mild Alzheimer's disease dementia by the NIA-AA core clinical criteria for probable Alzheimer's disease dementia. In some embodiments, a subject has been diagnosed with mild Alzheimer's disease dementia by a CDR score of 0.5 to 1.0 and a Memory Box score of 0.5 or greater before treatment.

In some embodiments, one or more doses of an anti-beta-amyloid antibody administered to a human subject comprise one or more doses of an anti-beta-amyloid antibody selected from: aducanumab, lecanemab, gantenerumab, donanemab, LY3002813, bapineuzumab, crenezumab, MEDI-1814, and solanezumab.

In some embodiments, an anti-beta-amyloid antibody comprises a VHCDR1 with the amino acid sequence of SEQ ID NO:3, a VHCDR2 with the amino acid sequence of SEQ ID NO: 4, and a VHCDR3 with the amino acid sequence of SEQ ID NO:5, and a VLCDR1 with the amino acid sequence of SEQ ID NO:6, a VLCDR2 with the amino acid sequence of SEQ ID NO: 7, and a VLCDR3 with the amino acid sequence of SEQ ID NO:8. In some embodiments, an anti-beta-amyloid antibody comprises a VH comprising the amino acid sequence of SEQ ID NO: 1 and a VL comprising the amino acid sequence of SEQ ID NO:2. In some embodiments, an anti-beta-amyloid antibody comprises a heavy chain comprising the amino acid sequence of SEQ ID NO:10 and a light chain comprising the amino acid sequence of SEQ ID NO: 11. In some embodiments, an anti-beta-amyloid antibody is aducanumab.

In some embodiments, administering an anti-beta-amyloid antibody is by intravenous administration. In some embodiments, administering an anti-beta-amyloid antibody is by subcutaneous administration.

In some embodiments, provided methods comprise administering a dose of the anti-beta-amyloid antibody in an amount of 3 mg antibody/kg of body weight of the human subject. In some embodiments, provided methods comprise administering a dose of the anti-beta-amyloid antibody in an amount of 6 mg antibody/kg of body weight of the human subject. In some embodiments, provided methods comprise administering a dose of the anti-beta-amyloid antibody in an amount of 10 mg antibody/kg of body weight of the human subject.

In some embodiments, provided methods comprise administering the anti-beta-amyloid antibody as a multiple dose regimen comprising multiple doses of 1 mg antibody/kg of body weight of the human patient are administered to the human patient at intervals of 4 weeks, multiple doses of 3 mg antibody/kg of body weight of the human patient are administered to the human patient at intervals of 4 weeks, multiple doses of 6 mg antibody/kg of body weight of the human patient are administered to the human patient at intervals of 4 weeks, and multiple doses of 10 mg antibody/kg of body weight of the human patient are administered to the human patient at intervals of 4 weeks.

In some embodiments, provided methods comprise (a) administering a dose of the anti-beta-amyloid antibody to the human subject in an amount of 1 mg antibody/kg of body weight of the human subject; (b) 4 weeks after step (a), administering a dose of the antibody to the human subject in an amount of 1 mg antibody/kg of body weight of the human subject; (c) 4 weeks after step (b), administering a dose of the antibody to the human subject in an amount of 3 mg antibody/kg of body weight of the human subject; (d) 4 weeks after step (c), administering a dose of the antibody to the human subject in an amount of 3 mg antibody/kg of body weight of the human subject; (e) 4 weeks after step (d), administering a dose of the antibody to the human subject in an amount of 6 mg antibody/kg of body weight of the human subject; (f) 4 weeks after step (e), administering a dose of the antibody to the human subject in an amount of 6 mg antibody/kg of body weight of the human subject; and (g) in consecutive intervals of 4 weeks after step (f), administering a dose of the antibody to the human subject in an amount of 10 mg antibody/kg of body weight of the human subject.

In some embodiments, provided methods comprise administering a dose of the antibody at a cumulative dose of at least 150 mg antibody/kg of body weight of the human subject. In some embodiments, provided methods comprise administering a dose of the antibody at a cumulative dose of at least 200 mg antibody/kg of body weight of the human subject. In some embodiments, provided methods comprise administering a dose of the antibody in an amount of 10 mg antibody/kg of body weight of the human subject every 4 weeks over at least 52 weeks. In some embodiments, provided methods comprise administering a dose of the antibody in an amount of 6 mg antibody/kg of body weight of the human subject every 4 weeks over at least 112 weeks. In some embodiments, provided methods comprise administering the antibody to the human subject in multiple doses and wherein the multiple doses comprise (a) at least two doses of 3 mg antibody/kg of body weight of the human subject every 4 weeks; and (b) at least thirty doses of 6 mg antibody/kg of body weight of the human subject every 4 weeks.

In some embodiments, a human subject is an ApoE carrier. In some embodiments, a human subject has Down's syndrome. In some embodiments, a human subject is aged ≥65 years. In some embodiments, a human subject is aged 65 to 74 years. In some embodiments, a human subject is aged ≥75 years.

In some embodiments, a human subject is aged 65 to 74 years, and said subject has a plasma p-tau level at a second time point that is reduced at least 5% to at least 20% relative to a first, baseline time point. In some embodiments, a human subject is aged 65 to 74 years, and said subject has a plasma p-tau level at a second time point that is reduced at least 10%, at least 11%, at least 12%, at least 13%, at least 14%, or at least 15% relative to a first, baseline time point. In some embodiments, a human subject is aged 65 to 74 years, and said subject has a plasma p-tau level that is reduced at least 10% to at least 30% relative to a placebo group of patients at the same time point. In some embodiments, a human subject is aged 65 to 74 years, and said subject has a plasma p-tau level that is reduced at least 15%, at least 16%, at least 17%, at least 18%, at least 19%, at least 20%, at least 21%, at least 22%, at least 23%, at least 24%, or at least 25% relative to a placebo group of patients at the same time point.

In some embodiments, a p-tau level in plasma of a human subject has an adjusted mean change from baseline that is reduced at least −0.2, −0.25, −0.3, −0.35, −0.4, −0.45, −0.5, −0.55, −0.6, −0.65, −0.7, −0.75, −0.8, −0.85, −0.9, −0.95, relative to the adjusted mean change from baseline of a placebo group of patients at the same time point. In some embodiments, the time point is after a period of 56 weeks, 78 weeks, 104 weeks, 128 weeks, 134 weeks, or longer.

In some embodiments, after 56 weeks of administering, a p-tau level in plasma of a human subject has an adjusted mean change from baseline that is reduced within a range of −0.1 to −0.6 relative to the adjusted mean change from baseline of a placebo group of patients at the same time point.

In some embodiments, after 78 weeks of administering, a p-tau level in plasma of a human subject has an adjusted mean change from baseline that is reduced within a range of −0.2 to −0.7 relative to the adjusted mean change from baseline of a placebo group of patients at the same time point.

In some embodiments, a human subject is aged ≥75 years, and said subject has a plasma p-tau level at a second time point that is reduced at least 5% to at least 25% relative to a first, baseline time point. In some embodiments, a human subject is aged ≥75 years, and said subject has a plasma p-tau level at a second time point that is reduced at least 10%, at least 11%, at least 12%, at least 13%, at least 14%, at least 15%, at least 16%, at least 17%, at least 18%, at least 19%, or at least 20% relative to a first, baseline time point. In some embodiments, a human subject is aged ≥75 years, and said subject has a plasma p-tau level that is reduced at least 20% to at least 40% relative to a placebo group of patients at the same time point. In some embodiments, a human subject is aged ≥75 years, and said subject has a plasma p-tau level that is reduced at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, or at least 35% relative to a placebo group of patients at the same time point.

In some embodiments, a human subject is an ApoE noncarrier, and said subject has a plasma p-tau level at a second time point that is reduced at least 5% to at least 25% relative to a first, baseline time point. In some embodiments, a human subject is an ApoE noncarrier, and said subject has a plasma p-tau level at a second time point that is reduced at least 10%, at least 11%, at least 12%, at least 13%, at least 14%, at least 15%, at least 16%, at least 17%, at least 18%, at least 19%, or at least 20% relative to a first, baseline time point. In some embodiments, a human subject is an ApoE noncarrier, and said subject has a plasma p-tau level that is reduced at least 10% to at least 40% relative to a placebo group of patients at the same time point. In some embodiments, a human subject is an ApoE noncarrier, and said subject has a plasma p-tau level that is reduced at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, or at least 35% relative to a placebo group of patients at the same time point.

In some embodiments, a human subject is an ApoE carrier, and said subject has a plasma p-tau level at a second time point that is reduced at least 5% to at least 20% relative to a first, baseline time point. In some embodiments, a human subject is an ApoE carrier, and said subject has a plasma p-tau level at a second time point that is reduced at least 5%, at least 6%, at least 7%, at least 8%, at least 9%, at least 10%, at least 11%, at least 12%, at least 13%, at least 14%, or at least 15% relative to a first, baseline time point. In some embodiments, a human subject is an ApoE carrier, and said subject has a plasma p-tau level that is reduced at least 10% to at least 30% relative to a placebo group of patients at the same time point. In some embodiments, a human subject is an ApoE carrier, and said subject has a plasma p-tau level that is reduced at least 15%, at least 16%, at least 17%, at least 18%, at least 19%, at least 20%, at least 21%, at least 22%, at least 23%, at least 24%, or at least 25% relative to a placebo group of patients at the same time point.

In some embodiments, a human subject does not develop an Amyloid Related Imaging Abnormality (ARIA) during the course of treatment that requires suspension of treatment.

In some embodiments, provided methods include methods of treating Alzheimer's Disease (AD) in the human subject. In some embodiments, provided methods include methods of monitoring treatment response in the human subject. In some embodiments, provided methods include methods of determining a treatment plan for the human subject. In some embodiments, provided methods include methods of monitoring the cognitive state of the human subject.

In some embodiments, provided methods include methods of characterizing or assessing efficacy of an anti-beta-amyloid antibody in a human subject.

In some embodiments, the present disclosure provides methods comprising determining a p-tau level in plasma of a human subject who has received at least one dose of an anti-beta amyloid antibody, and adjusting the amount of an anti-beta-amyloid antibody in a dose administered to the human subject based on the determined plasma p-tau level.

In some embodiments, the present disclosure provides methods for selecting a patient for treatment with an anti-beta-amyloid antibody comprising determining a p-tau level in plasma of a human subject.

In some embodiments, the present disclosure provides methods for determining a dose of an anti-beta-amyloid antibody to be administered to a human subject comprising determining a p-tau level in plasma of a human subject.

In some embodiments, the present disclosure provides methods for selecting a patient receiving an anti-beta-amyloid antibody for cognitive support services comprising determining a p-tau level in plasma of a human subject.

In some embodiments, the present disclosure provides methods comprising determining a p-tau level in plasma of a human subject who has received at least one dose of an anti-beta-amyloid antibody. In some embodiments, provided methods comprise comparing the p-tau level in plasma of the human subject to a reference plasma p-tau level. In some embodiments, a reference plasma p-tau level is a p-tau level in plasma of the human subject prior to receiving a dose of an anti-beta-amyloid antibody. In some embodiments, a reference plasma p-tau level is an average p-tau level in plasma of a plurality of human subjects.

In some embodiments, a plurality of human subjects are a plurality of healthy human subjects with no evidence of AD. In some embodiments, a plurality of human subjects are a plurality of human subjects with a known level of amyloid plaques. In some embodiments, a plurality of human subjects are a plurality of human subjects with a known level of tau tangles. In some embodiments, a plurality of human subjects are a plurality of human subjects with a predetermined level of cognition.

In some embodiments, a human subject is classified as being susceptible to worsening cognition if the p-tau level in plasma of the human subject is higher than a reference plasma p-tau level. In some embodiments, a human subject is classified as being susceptible to increased numbers of amyloid plaques if the p-tau level in plasma of the human subject is higher than a reference plasma p-tau level. In some embodiments, a human subject is classified as being susceptible to increased numbers of tau tangles if the p-tau level in plasma of the human subject is higher than a reference plasma p-tau level.

In some embodiments, provided methods comprise administering another dose of the anti-beta-amyloid antibody to the human subject if the p-tau level in plasma of the human subject is higher than a reference plasma p-tau level. In some embodiments, administering another dose of the anti-beta-amyloid antibody to the human subject comprises administering a dose of the anti-beta-amyloid antibody to the human subject that is equal to or greater than a prior dose of the anti-beta-amyloid antibody received by the human subject.

In some embodiments, a p-tau level in plasma comprises tau with phosphorylation at one or more sites selected from: Y18, S46, T50, S69, T71, S113, T123, T153, T175, T181, S184, S85, S191, Y197, S198, S199, S202, T205, S208, S210, T212, S214, T217, T231, S235, S237, S238, S258, S262, S289, S356, Y397, S400, T403, S404, S409, S412, S413, T414/S416, S422, T427, S433, and S435. In some embodiments, a p-tau level in plasma comprises p-tau¹⁸¹ and/or p-tau²¹⁷. In some embodiments, the p-tau level is a plasma p-tau¹⁸¹ level.

In some embodiments, a human subject is an ApoE carrier. In some embodiments, a human subject has Down's syndrome. In some embodiments, a human subject is aged ≥65 years. In some embodiments, a human subject is aged 65 to 74 years. In some embodiments, a human subject is aged ≥75 years.

In some embodiments, the present disclosure provides kits comprising a composition comprising an anti-beta-amyloid antibody for administration to a human subject and instructions for use. In some embodiments, the instructions direct administration to a human subject that has one or more characteristics.

In some embodiments, the instructions direct administration to a human subject that has Down's syndrome. In some embodiments, the instructions direct administration to a human subject that is aged ≥65 years. In some embodiments, the instructions direct administration to a human subject that is aged 65 to 74 years. In some embodiments, the instructions direct administration to a human subject that is aged ≥75 years.

In some embodiments, the instructions direct administration to a human subject that has a plasma p-tau level is above a reference or threshold level. In some embodiments, a p-tau level in plasma comprises p-tau¹⁸¹ and/or p-tau²¹⁷. In some embodiments, the p-tau level is a plasma p-tau¹⁸¹ level.

In some embodiments, kits for use in accordance with the present disclosure may include, instructions for processing samples, performing tests on samples, instructions for interpreting the results, etc.

In some embodiments, the instructions to determine a p-tau level in plasma of a human subject at a first and second time point. In some embodiments, a first time point for determining a p-tau level is prior to the human subject receiving a dose of the anti-beta-amyloid antibody. In some embodiments, a second time point for determining a p-tau level is after the human subject has received at least one dose of the anti-beta-amyloid antibody. In some embodiments, a second time point for determining the level of plasma p-tau is after at least 10 doses of anti-beta-amyloid antibody have been administered to the human subject. In some embodiments, a second time point for determining the level of plasma p-tau is after at least 14 doses of anti-beta-amyloid antibody have been administered to the human subject. In some embodiments, a second time point for determining the level of plasma p-tau is after at least 19 doses of anti-beta-amyloid antibody have been administered to the human subject.

In some embodiments, the instructions direct continued administration to a human subject whose change in plasma p-tau level between the first and second time point meets or exceeds a reference or threshold level. In some embodiments, a p-tau level in plasma comprises p-tau¹⁸¹ and/or p-tau²¹⁷. In some embodiments, the p-tau level is a plasma p-tau¹⁸¹ level.

These and other features and advantages provided in the present disclosure will be more fully understood from the following detailed description taken together with the accompanying claims. It is noted that the scope of the claims is defined by the recitations therein and not by the specific discussion of features and advantages set forth in the present description.

### BRIEF DESCRIPTIONS OF THE DRAWINGS

The Drawing included herein, which is composed of the following Figures, is for illustration purposes only and not for limitation.

FIG. 1 depicts a schematic of a study design for dosing an exemplary anti-Abeta (Aβ) antibody, such as, e.g., aducanumab.

FIG. 2 shows the change from Baseline in Aβ PET Composite standardized uptake value ratio (SUVR) by MMRM of ¹⁸F-florbetapir amyloid PET analysis population of Study 1. ***p<0.0001 compared with placebo (nominal).

FIG. 3 shows the change from Baseline in Aβ PET Composite SUVR by MMRM of ¹⁸F-florbetapir Aβ PET Analysis Population of Study 2. ***p<0.0001 compared with placebo (nominal).

FIG. 4 depicts the adjusted mean change in plasma p-tau¹⁸¹ levels of the population of Study 1.

FIG. 5 depicts the adjusted mean change in plasma p-tau¹⁸¹ levels of the population of Study 2.

FIG. 6 depicts scatterplots of change from baseline plasma p-tau¹⁸¹ vs change from baseline florbetapir amyloid PET composite SUVR (reference region=cerebellum) for the Population of Study 1 at Week 78. Circles represent subjects receiving placebo; squares represent subjects receiving a “low dose” of an exemplary anti-Aβ antibody treatment; and triangles represent subjects receiving a “high dose” of an exemplary anti-Aβ antibody treatment.

FIG. 7 depicts scatterplots of change from baseline plasma p-tau¹⁸¹ vs change from baseline florbetapir amyloid PET composite SUVR (reference region=cerebellum) for the Population of Study 2 at Week 78. Circles represent subjects receiving placebo; squares represent subjects receiving a “low dose” of an exemplary anti-Aβ antibody treatment; and triangles represent subjects receiving a “high dose” of an exemplary anti-Aβ antibody treatment.

FIG. 8A and FIG. 8B depict change from baseline plasma p-tau¹⁸¹ in subjects whose baseline florbetapir amyloid PET composite SUVR was greater than >1.10 (open circles, dashed line) and in subjects whose baseline florbetapir amyloid PET composite SUVR was ≤1.10 (solid circles, solid line), for the populations of patients from Study 1 and Study 2, respectively.

FIG. 9 depicts Forest plots of plasma p-tau¹⁸¹ subgroup analysis for subjects receiving a “low dose” of an exemplary anti-Aβ antibody treatment.

FIG. 10 depicts Forest plots of plasma p-tau¹⁸¹ subgroup analysis for subjects receiving a “high dose” of an exemplary anti-Aβ antibody treatment.

FIG. 11 depicts Forest plots of plasma p-tau¹⁸¹ subgroup analysis based on baseline amyloid PET SUVR for subjects receiving a “high dose” of an exemplary anti-Aβ antibody treatment.

FIG. 12 depicts Forest plots of plasma p-tau¹⁸¹ subgroup analysis based on baseline amyloid PET SUVR for subjects receiving a “low dose” of an exemplary anti-Aβ antibody treatment.

FIG. 13 depicts Forest plots of plasma p-tau¹⁸¹ subgroup analysis based on baseline plasma p-tau¹⁸¹ for subjects receiving a “high dose” of an exemplary anti-Aβ antibody treatment.

FIG. 14 depicts Forest plots of plasma p-tau¹⁸¹ subgroup analysis based on baseline plasma p-tau¹⁸¹ for subjects receiving a “low dose” of an exemplary anti-Aβ antibody treatment.

FIG. 15 depicts line plots of plasma p-tau¹⁸¹ adjusted mean change from baseline over time for subjects in Study 1 over the placebo-controlled and long term extension periods.

FIG. 16 depicts line plots of plasma p-tau¹⁸¹ adjusted mean change from baseline over time for subjects in Study 2 over the placebo-controlled and long term extension periods.

### DETAILED DESCRIPTION

Aducanumab is a human monoclonal antbody targeting Aβ aggregates. In June 2021, the U.S. Food and Drug Administration (FDA) granted accelerated approval for ADUHELM™ (aducanumab-avwa) as the first and only Alzheimer's disease treatment to address a defining pathology of the disease by reducing amyloid beta plaques in the brain. The efficacy of ADUHELM was evaluated in two Phase 3 clinical trials—EMERGE (Study 1) and ENGAGE (Study 2)—in patients with early stages of Alzheimer's disease (mild cognitive impairment and mild dementia) with confirmed presence of amyloid pathology. The effects of ADUHELM were also assessed in the double-blind, randomized, placebo-controlled, dose-ranging Phase 1b study, PRIME (Study 3). In these studies, ADUHELM consistently showed a dose- and time-dependent effect on the lowering of amyloid beta plaques (by 59 percent [p<0.0001] in ENGAGE, 71 percent [p<0.0001] in EMERGE, and 61 percent [p<0.0001] in PRIME). Using data from the phase 3 aducanumab trials, EMERGE and ENGAGE, the present disclosure describes investigation of the effect of aducanumab treatment on plasma p-tau¹⁸¹ in ˜7,000 plasma samples from 1815 patients with early Alzheimer's disease.

### Certain Definitions

As utilized in accordance with the present disclosure, the following terms, unless otherwise indicated, shall be understood to have the following meanings. Unless otherwise required by context, singular terms shall include pluralities and plural terms shall include the singular.

Additionally, singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “about” or “approximately” includes being within a meaningful range of a value. The allowable variation encompassed by the term “about” or “approximately” depends on the particular system under study, and can be readily appreciated by one of ordinary skill in the art.

As used herein, the term “administering” or “administration” typically refers to the administration of a composition (e.g., comprising an anti-Aβ antibody as described herein) to a subject to achieve delivery of an agent that is, or is included in, a composition to a target site or a site to be treated. Those of ordinary skill in the art will be aware of a variety of routes that may, in appropriate circumstances, be utilized for administration to a subject, e.g., a human subject. For example, in some embodiments, administration may be parenteral. In some embodiments, administration may involve a single dose or multiple doses. In some embodiments, administration may involve application of a fixed number of doses. In some embodiments, administration may involve dosing that is intermittent (e.g., a plurality of doses separated in time) and/or periodic (e.g., individual doses separated by a common period of time) dosing.

The term “determining” as used herein can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information. In some embodiments, determining involves receiving relevant information and/or materials from a source. In some embodiments, determining involves comparing one or more features of a sample or entity to a comparable reference.

As used herein, the terms “improve”, “increase”, “inhibit’, “reduce”, or grammatical equivalents thereof, indicate values that are relative to a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

### Alzheimer's Disease

The present disclosure provides methods for treating patients having or suspected of having Alzheimer's disease, abbreviated herein as ΔD. AD is a dementia that is primarily identified by clinical diagnosis and established by certain markers of the disease.

AD is a continuum having certain operationally defined stages of disease progression. AD pathology begins prior to the onset of clinical symptoms. For example, amyloid plaques, one marker of AD pathology, form 10-20 years prior to the onset of AD dementia. The currently recognized stages of AD include preclinical, prodromal, mild, moderate, and severe. These stages may be further divided into subcategories based on the severity of symptoms and measures of AD progression.

Because AD does not occur in discrete stages, those skilled in the art will recognize that the differences between patient groups may not be distinct in a particular clinical setting. Nevertheless, the clinical disease stage can be characterized by measures, and changes in these measures over time, such as Aβ accumulation (CSF/PET), synaptic dysfunction (FDG-PET/fMRI), tau-mediated neuronal injury (CSF), brain structure (volumetric MRI), cognition, and clinical function. (Jack C R, et al. Hypothetical model of dynamic biomarkers of the Alzheimer's pathological cascade. Lancet Neurol., 2010; 9 (1): 119-28).

Current core clinical criteria for all dementia, referred to as the NINCDS-ADRDA criteria (McKhann G M, V. diagnosis of dementia due to Alzheimer's disease: Recommendations from the National Inst. on Aging-Alzheimer's Association workgroups on diagnostic guidelines for Alzheimer's disease. Alzheimer's & Dementia, 7 (2011) 263-269), are known in the art and can be employed in practicing this invention. They include cognitive or behavioral impairment involving impaired ability to acquire and remember new information, impaired reasoning and handling of complex tasks, impaired visuospatial abilities, impaired language functions (speaking, reading, writing), and changes in personality, behavior, or comportment. Alzheimer's disease is currently diagnosed using the core criteria and is typically characterized by symptoms which have a gradual onset over months to years, not sudden over hours or days (insidious onset). There is usually a clear-cut history of worsening of cognition by report or observation in Alzheimer's disease subjects.

Other diagnostic classification systems have evolved as new information on AD has become available. These systems include the International Working Group (IWG) new research criteria for diagnosis of AD (Dubois B et al., Lancet Neurol., 2007; 6 (8): 734-736), IWG research criteria, (Dubois et al., Lancet Neurol., 2010; 9 (11): 1118-27), NIA/AA Criteria (Jack C R et al. Alzheimer's Dement., 2011; 7 (3): 257-62), and DSM-5 criteria (American Psychiatric Association, DSM-5, 2013). These classification systems can also be employed in diagnosing AD subjects for treatment according to the methods of this disclosure.

In some embodiments, wherein Alzheimer's disease is mild Alzheimer's disease, early Alzheimer's disease, prodromal Alzheimer's disease, mild Alzheimer's disease dementia, mild cognitive impairment due to Alzheimer's disease, mid-stage Alzheimer's disease, or late-stage Alzheimer's disease.

### Patients

As used herein, the term “patient” includes any human subject who may benefit from therapy with an anti-beta-amyloid antibody. In some embodiments, a patient has or is suspected of having a disease associated with the accumulation and deposition of beta amyloid, such as Alzheimer's disease, Down's syndrome, mild cognitive impairment, cerebral amyloid angiopathy, vascular dementia, and/or multi-infarct dementia.

In some embodiments, a patient is one for whom diagnosis, prognosis, prevention, or therapy for Alzheimer's disease is desired, and includes a human subject in need of treatment. Those in need of treatment include those already with AD, as well as those prone to have AD, or those in which the manifestation of AD is to be prevented. Typical patients will be men or women aged 40 to 90 (e.g., 45 to 90, 50 to 90, 55 to 90, 60 to 90). In some embodiments, a patient is aged ≥65 years. In some embodiments, a patient is aged 65 to 74 years. In some embodiments, a patient is aged ≥75 years.

In some embodiments, the disclosure provides a method of treating a patient with AD (including, without limitation, patients with preclinical, prodromal, mild, moderate, or severe AD). In certain instances, the disclosure provides a method of treating a patient with prodromal Alzheimer's disease. In some instances, the disclosure provides a method of treating a patient with early Alzheimer's disease. In some instances, the disclosure provides a method of treating a patient to reduce clinical decline in Alzheimer's disease. In some embodiments, a patient with Alzheimer's disease also has Down's syndrome.

In some instances, the disclosure provides a method of treating a patient with mild cognitive impairment due to Alzheimer's disease. In other instances, the disclosure provides a method of treating a patient with mild Alzheimer's disease dementia. In some embodiments, the patient has amyloid pathology confirmed, e.g., by positron emission tomography (PET) imaging. In some cases, amyloid β pathology is confirmed by [¹⁸F]-florbetapir PET imaging. In some cases, amyloid β pathology is confirmed by [¹⁸F]-flutemetomol PET imaging. In some cases, amyloid β pathology is confirmed by [¹⁸F]-florbetaben PET imaging. In some cases, amyloid β pathology is confirmed by CSF amyloid β analysis. In some cases, amyloid β pathology is confirmed by blood amyloid β analysis. In some cases, amyloid β pathology is confirmed by Congo red staining and birefringence under polarized microscopy. In some cases, amyloid β pathology is confirmed by immunohistochemistry (IHC), electron microscopy, or mass spectrometry. In some cases, amyloid β pathology is confirmed by any method to assess levels of amyloid β.

In some instances, the disclosure provides a method of treating a patient with Down's syndrome that also has or is suspected of having an accumulation of beta amyloid deposits. In some embodiments, a patient with Down's syndrome has a confirmed amyloid pathology. In some embodiments, a patient with Down's syndrome has been diagnosed with AD.

In certain instances, a patient to be treated has an mini-mental state examination (MMSE) score between 24-30 (inclusive). In some instances, the patient to be treated has a CDR global score of 0.5. In some instances, the patient to be treated has a RBANS score of less than or equal to 85 (based upon Delayed Memory Index score). In some instances, the patient to be treated has at least 6 years of work experience. In some cases, the patient to be treated has an MMSE score between 24-30 (inclusive); a CDR global score of 0.5; and a RBANS score of less than or equal to 85 (based upon Delayed Memory Index score). In certain instances, the patient is an ApoE carrier (e.g., ApoE4 positive). In certain instances, the patient is an ApoE noncarrier (e.g., ApoE4 negative).

In some embodiments, the patient to be treated has mid-stage Alzheimer's disease, characterized by a MMSE score of about 10-20 or equivalent score on other scales. In some embodiments, late-stage Alzheimer's disease is characterized by an MMSE score of about 9 or less or equivalent score on other scales.

In some embodiments, a patient has p-tau tangles, p-tau threads, and/or p-tau neuritic plaques. In some embodiments, a patient has neocortical p-tau tangles, neocortical p-tau threads, and/or neocortical p-tau neuritic plaques. In some embodiments, p-tau tangles, p-tau threads, and/or p-tau neuritic plaques are detected in a patient by positron emission tomography (PET) scanning of the human subject's brain. In some embodiments, p-tau tangles, p-tau threads, and/or p-tau neuritic plaques are detected in a patient by analysis of the amount of p-tau and/or t-tau in the human subject's CSF and/or plasma.

AD patients in need of treatment range from subjects with amyloid pathology and early neuronal degeneration to subjects with widespread neurodegeneration and irreversible neuronal loss with progressive cognitive and functional impairment to subjects with dementia.

Patients with preclinical AD can be identified by asymptomatic stages with or without memory complaints and emerging episodic memory and executive function deficits. This stage is typically characterized by the appearance of in vivo molecular biomarkers of AD and the absence clinical symptoms.

Prodromal AD patients are pre-dementia stage characterized predominantly by cognitive deficits and emerging functional impairment with disease progression. Prodromal AD patients typically have mini-mental state examination (MMSE) scores between 24-30 (inclusive), a spontaneous memory complaint, objective memory loss defined as a free recall score of <27 on the Free and Cued Selective Reminding Test (FCSRT), a global Clinical Dementia Rating (CDR) score of 0.5, absence of significant levels of impairment in other cognitive domains, and essentially preserved activities of daily living, and an absence of dementia.

Patients with mild AD typically have MMSE scores between 20-26 (inclusive), a global CDR of 0.5 or 1.0, and meet the National Institute on Aging-Alzheimer's Association core clinical criteria for probable AD (see Section 22).

Basing AD diagnosis on clinical symptoms, mild stage AD patients will exhibit conspicuous behavior at work, forgetfulness, mood swings, and attention disturbances. Moderate stage AD patients will exhibit cognitive deficits, restricted everyday activities, orientation disturbance, apraxia, agnosia, aphasia, and behavioral abnormalities. Severe stage AD patients are characterized by loss of independence, decay of memory and speech, and incontinence.

In certain embodiments, treatment is of earlier-stage patients who are amyloid positive as assessed by [¹⁸F]-florbetapir PET scans. In certain embodiments, treatment is of earlier-stage patients who are amyloid positive as assessed by ¹⁸F-flutemetomol PET scans. In certain embodiments, treatment is of earlier-stage patients who are amyloid positive as assessed by 18F-florbetaben PET scans. In certain instances, the human subject is confirmed to have a brain amyloid beta pathology prior to the initiation of treatment. The patient may be asymptomatic for, or exhibit only transient symptoms of, headache, confusion, gait difficulties, or visual disturbances. The patient may or may not be an ApoE carrier (e.g., ApoE4 carrier) as determined by ApoE genotyping.

In other embodiments, treatment is of patients having any medical or neurological condition (other than AD) that might be a contributing cause of the subject's cognitive impairment, such as stroke or other cerebrovascular condition, other neurodegenerative disease, a history of clinically significant psychiatric illness, acute or sub-acute micro- or macro hemorrhage, prior macrohemorrhage, or superficial siderosis. These patients can be treated following screening and selection by a qualified clinician.

### Anti-Aβ Antibodies

There are numerous anti-beta amyloid antibodies known in the art, including, for example, aducanumab, lecanemab, gantenerumab, donanemab, LY3002813, bapineuzumab, crenezumab, MEDI-1814, and solanezumab.

In some embodiments, an anti-beta amyloid antibody used in a method of the present disclosure is or comprises lecanemab. In some embodiments, an anti-beta amyloid antibody used in a method of the present disclosure is or comprises gantenerumab. In some embodiments, an anti-beta amyloid antibody used in a method of the present disclosure is or comprises donanemab. In some embodiments, an anti-beta amyloid antibody used in a method of the present disclosure is or comprises LY3002813. In some embodiments, an anti-beta amyloid antibody used in a method of the present disclosure is or comprises bapineuzumab. In some embodiments, an anti-beta amyloid antibody used in a method of the present disclosure is or comprises crenezumab. In some embodiments, an anti-beta amyloid antibody used in a method of the present disclosure is or comprises MEDI-1814. In some embodiments, an anti-beta amyloid antibody used in a method of the present disclosure is or comprises solanezumab.

In some embodiments, an anti-beta amyloid antibody used in a method of the present disclosure comprises aducanumab (also known as BIIB037). Aducanumab is an anti-Aβ antibody that recognizes aggregated forms of Aβ, including plaques. BIIB037 contains a human kappa light chain. BIIB037 consists of 2 heavy and 2 human kappa light chains connected by inter-chain disulfide bonds. By “BIIB037” or “aducanumab” is meant an anti-Aβ antibody comprising the amino acid sequences set forth in SEQ ID NOs: 10 and 11.

In vitro characterization studies have established that antibody BIIB037 recognizes a conformational epitope present in Aβ aggregates, the accumulation of which is believed to underlie the development and progression of AD.

In vivo pharmacology studies indicate that a murine IgG2a chimeric version of the antibody (ch 12F6A) with similar properties significantly reduces amyloid plaque burden in the brains of aged Tg2576 mice, a mouse model of AD. The reduction in parenchymal amyloid was not accompanied by a change in vascular amyloid, as has been reported for certain anti-Aβ antibodies (Wilcock O M, Colton C A. Immunotherapy, vascular pathology, and microhemorrhages in transgenic mice. CNS & Neurological Disorders Drug Targets, 2009 March; 8 (1): 50-64).

The VH and VL of antibody BIIB037 have amino acid sequences that are identical to the amino acid sequence of the VH and VL of antibody NI-101.12F6A described in U.S. Pat. No. 8,906,367 (see, Tables 2-4; incorporated by reference in its entirety herein). Specifically, antibody BIIB037 has an antigen binding domain comprising VH and VL variable regions depicted in Table A (VH) and Table B (VL), corresponding complementarity determining regions (CDRs) depicted in Table C, and heavy and light chains depicted in Table D (H) and Table E (L).

TABLE A
Amino acid sequences of the V_{H} region of anti-Aβ
antibody BIIB037 (VH CDRs
(Kabat definition) underlined).
Variable heavy chain sequence

QVQLVESGGG VVQPGRSLRL SCAASGFAFS SYGMHWVRQA
PGKGLEWVAV IWFDGTKKYY TDSVKGRFTI SRDNSKNTLY
LQMNTLRAED TAVYYCARDR GIGARRGPYY MDVWGKGTTV
TVSS (SEQ ID NO: 1)

TABLE B
Amino acid sequences of the V_{L} region of anti-Aβ
antibody BIIB037 (V_{L} CDRs (Kabat
definition) underlined).
Variable light chain sequence (kappa or lambda)

DIQMTQSPSS LSASVGDRVT ITCRASQSIS SYLNWYQQKP
GKAPKLLIYA ASSLQSGVPS RFSGSGSGTD FTLTISSLQP
EDFATYYCQQ SYSTPLTFGG GTKVEIKR (SEQ ID NO: 2)

TABLE C
Denomination of CDR protein sequences in Kabat
Nomenclature of V_{H} and V_{L} regions of anti-Aß
antibody BIIB037.

| CDR | Variable heavy chain | Variable light chain |
|---|---|---|
| CDR1 | SYGMH | RASQSISSYLN |
| | (SEQ ID NO: 3) | (SEQ ID NO: 6) |
| CDR2 | VIWFDGTKKYYTDSVKG | AASSLQS |
| | (SEQ ID NO: 4) | (SEQ ID NO: 7) |
| CDR3 | DRGIGARRGPYYMDV | QQSYSTPLT |
| | (SEQ ID NO: 5) | (SEQ ID NO: 8) |

The amino acid sequence of the mature heavy chain of BIIB037 is provided in Table D below.

TABLE D
Amino acid sequences of the heavy chain of
anti-Aβ antibody BIIB037 (heavy chain CDRs
(Kabat definition) underlined).
Heavy chain sequence

QVQLVESGGG VVQPGRSLRL SCAASGFAFS SYGMHWVRQA
PGKGLEWVAV IWFDGTKKYY TDSVKGRFTI SRDNSKNTLY
LQMNTLRAED TAVYYCARDR GIGARRGPYY MDVWGKGTTV
TVSSASTKGP SVFPLAPSSK STSGGTAALG CLVKDYFPEP
VTVSWNSGAL TSGVHTFPAV LQSSGLYSLS SVVTVPSSSL
GTQTYICNVN HKPSNTKVDK RVEPKSCDKT HTCPPCPAPE
LLGGPSVFLF PPKPKDTLMI SRTPEVTCVV VDVSHEDPEV
KFNWYVDGVE VHNAKTKPRE EQYNSTYRVV SVLTVLHQDW
LNGKEYKCKV SNKALPAPIE KTISKAKGQP REPQVYTLPP
SREEMTKNQV SLTCLVKGFY PSDIAVEWES NGQPENNYKT
TPPVLDSDGS FFLYSKLTVD KSRWQQGNVF SCSVMHEALH
NHYTQKSLSL SPG (SEQ ID NO: 10)

The amino acid sequence of the mature light chain of BIIB037 is provided in Table E below.

TABLE E
Amino acid sequences of the light chain of
anti-Aß antibody BIIB037 (light chain CDRs
(Kabat definition) underlined).
Light chain sequence

DIQMTQSPSS LSASVGDRVT ITCRASQSIS SYLNWYQQKP
GKAPKLLIYA ASSLQSGVPS RFSGSGSGTD FTLTISSLQP
EDFATYYCQQ SYSTPLTFGG GTKVEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC (SEQ ID NO: 11)

In addition to antibody BIIB037, this disclosure contemplates the use of the other anti-Abeta-amyloid antibodies, such as antibodies comprising either the VH region comprising or consisting of SEQ ID NO:1 or the VL region comprising or consisting of SEQ ID NO:2, or antibodies comprising the VH region comprising or consisting of SEQ ID NO: 1 and the VL region comprising or consisting of SEQ ID NO:2, wherein the VH and/or VL regions have one or more substitutions, deletions, and/or insertions. In some embodiments, these VH and VL regions may have up to 25, up to 20, up to 15, up to 10, up to 5, or 1, 2, 3, 4, 5, 6, 7, 8, 9, 10, 11, 12, 13, 14, 15, 16, 17, 18, 19, or 20 amino acid substitutions and still bind beta-amyloid. In specific embodiments, these amino acid substitutions occur only in the framework region. In some embodiments, the amino acid substitution(s) is/are conservative amino acid substitutions. In certain embodiments, the VH and VL regions may include 1 to 5 (1, 2, 3, 4, 5) amino acid deletions and/or additions and still bind beta-amyloid. In certain embodiments, these deletions and/or additions are made at the N- and/or C-terminus of the VH and/or VL regions. In some embodiments, one amino acid is deleted and/or added at the N and/or C-terminus of the VH region. In some embodiments, one amino acid is deleted and/or added at the N and/or C-terminus of the VL region.

Other antibodies contemplated for use in the disclosure include antibodies comprising the variable heavy chain (VH) CDRs and the variable light chain (VL) CDRs in Table C. Thus, the anti-beta amyloid antibodies comprise the CDRs comprising or consisting of the amino acid sequences of SEQ ID NOs.: 3-8. In some embodiments, the anti-beta amyloid antibodies comprise the CDRs comprising or consisting of the amino acid sequences of SEQ ID NOs.: 4-8 and include as VH CDR1 an amino acid sequence comprising or consisting of GFAFSSYGMH (SEQ ID NO:9). In some instances, the disclosure encompasses anti-Abeta-amyloid antibodies comprising the VH and VL CDRs of BIIB037 based on any CDR definition (e.g., Kabat, Chothia, enhanced Chothia, AbM, or contact definition). See, e.g., http://www.bioinf.org.uk/abs/index.html. In some embodiments, the disclosure encompasses anti-Abeta-amyloid antibodies comprising the VH and VL CDRs of BIIB037 based on the Chothia definition. In some embodiments, the disclosure encompasses anti-Abeta-amyloid antibodies comprising the VH and VL CDRs of BIIB037 based on the enhanced Chothia definition. In some embodiments, the disclosure encompasses anti-Abeta-amyloid antibodies comprising the VH and VL CDRs of BIIB037 based on the AbM definition. In yet another embodiment, the disclosure encompasses anti-Abeta-amyloid antibodies comprising the VH and VL CDRs of BIIB037 based on the contact definition.

Antibody BIIB037 and other antibodies employed in the invention can be prepared using known methods. In some embodiments, the antibody is expressed in a Chinese hamster ovary (CHO) cell line.

The maximum tolerated amount of the anti-Aβ antibody is that quantity of the antibody which will produce a clinically significant response in the treatment of Alzheimer's disease consistent with safety. A principal safety concern in treating patients according to the method of the invention is the occurrence of ARIA, especially ARIA-E or ARIA-H. The methods of the invention make it possible to employ higher doses of antibody BIIB037 for the treatment of patients for AD than was feasible using previously known protocols.

It will be understood that dose adjustments can be implemented during the treatment protocol. For example, for reasons of safety or efficacy, doses can be increased so that the effects of the anti-Aβ antibody on AD can be enhanced or doses can be decreased so that the ARIA rate and severity can be mitigated. If a dose is missed, the patient should preferably resume dosing by receiving the missed dose and continuing thereafter according to the described regimen.

In certain embodiments, the anti-Aβ antibody is administered to the patient by intravenous infusion following dilution into saline. When using this mode of administration, each infusion step in the titration regime of the invention will typically take about 1 hour.

The dose ranges and other numerical values herein include a quantity that has the same effect as the numerically stated amount as indicated by treatment of Alzheimer's disease in the patient and a reduction in the incidence or susceptibility of the patient to ARIA when compared to an individual not treated by the method of the invention. At the very least, each numerical parameter should be construed in light of the number of significant digits, applying ordinary rounding techniques. In addition, any numerical value inherently contains certain errors from the standard deviation of its measurement and such values are within the scope of the invention.

### Treatment

The present disclosure provides, among other things, methods and compositions for treatment of a disease associated with the accumulation and deposition of beta amyloid, such as Alzheimer's disease (AD), Down's syndrome, mild cognitive impairment, cerebral amyloid angiopathy, vascular dementia, and/or multi-infarct dementia. In some embodiments, a method of treatment of AD described herein includes: (a) inhibiting AD, e.g. arresting its development; (b) relieving AD, e.g. causing regression of AD; or (c) prolonging survival as compared to expected survival if not receiving treatment.

In some embodiments, the treatment is therapeutic. In some embodiments, treatment has a disease modifying effect. A disease modifying effect, as used herein, means that the treatment slows or delays the underling pathological or pathophysiological disease processes and/or there is an improvement in clinical signs and symptoms of AD relative to placebo.

In some embodiments, treatment results in symptomatic improvement. This may consist of enhanced cognition, more autonomy, and/or improvement in neuropsychiatric and behavioral dysfunction, even if for only a limited duration.

In some embodiments, the disclosure relates to methods for delaying clinical decline or progression of disease, or relief of symptoms. Delaying clinical decline or disease progression directly impacts the patient and care-givers. It delays disability, maintains independence, and allows the patient to live a normal life for a longer period of time. Relief of symptoms to the best degree possible can incrementally improve cognition, function, and behavioral symptoms, as well as mood.

The present disclosure describes, among other things, a titration regimen (sequential administration of increasing doses of an anti-beta amyloid antibody) to treat Alzheimer's disease. In some instances, the Alzheimer's disease is mild Alzheimer's disease, early Alzheimer's disease, prodromal Alzheimer's disease, mild Alzheimer's disease dementia, or mild cognitive impairment due to Alzheimer's disease.

In some provided methods of treatment of Alzheimer's disease, the anti-beta amyloid antibody is administered to a human patient in increasing amounts over a period of time. This procedure of sequentially administering the antibody to the patient is referred to herein as “titration” because it involves administering a standardized pharmaceutical of known concentrations in carefully measured amounts until completion of the procedure.

One of the advantages of the titration regimen of the invention is that it makes it possible to administer higher doses of the monoclonal antibody to AD patients without incurring the same extent of ARIA observed with a standard dose regimen. In certain embodiments, the higher dose comprises a dose or doses of the anti-Aβ antibody of 10 mg/kg of the body weight of the subject. Without intending to be limited to any particular mechanism, it is believed that titration results in lower initial amyloid removal and slower removal during the overall treatment.

Titration of the anti-Aβ antibody (e.g., BIIB037) is carried out in multiple doses. For example, two doses of the antibody can be administered to the patient in an amount per dose that is less than the minimum therapeutic amount, followed by 4 doses of the antibody in an amount per dose that is about equal to the minimum therapeutic amount. This regime can then be followed by multiple doses in an amount per dose that is more than the minimum therapeutic amount, but less than the maximum tolerated amount until there is an acceptable change in AD in the patient. For example, doses can be administered approximately 4 weeks apart over approximately 52 weeks (a total of 14 doses). Progress can be monitored by periodic assessment.

In some instances, the disclosure features a method for reducing tau or treating Alzheimer's disease by reducing Abeta and/or tau in a human patient in need thereof, the method comprising sequentially administering multiple doses of an anti-Aβ antibody (e.g., BIIB037) in increasing amounts over a period of time to the human patient, wherein multiple doses of 1 mg antibody/kg of body weight of the human patient are administered to the human patient at intervals of about 4 weeks; multiple doses of 3 mg antibody/kg of body weight of the human patient are administered to the human patient at intervals of about 4 weeks; multiple doses of 6 mg antibody/kg of body weight of the human patient are administered to the human patient at intervals of about 4 weeks; and multiple doses of 10 mg antibody/kg of body weight of the human patient are administered to the human patient at intervals of about 4 weeks. Multiple doses means at least two (e.g., 2, 3, 4, 5, 6, 7, 8, 9, 10, 11, 12, 123, 14, 15, 16, 17, 18, 19, 20, 21, 22, 23, 24, 25, 26, 27, 28, 29, 30) doses.

An exemplary protocol according to the disclosure, designated Protocol A, comprises:
(A) administering the anti-beta amyloid antibody to the patient in an amount of 1 mg/kg of body weight of the patient;
(B) 4 weeks after step (A), administering the anti-beta amyloid antibody to the patient in an amount of 1 mg/kg of body weight of the patient;
(C) 4 weeks after step (B), administering the anti-beta amyloid antibody to the patient in an amount of 3 mg/kg of body weight of the patient;
(D) 4 weeks after step (C), administering the anti-beta amyloid antibody to the patient in an amount of 3 mg/kg of body weight of the patient;
(E) 4 weeks after step (D), administering the anti-beta amyloid antibody to the patient in an amount of 6 mg/kg of body weight of the patient;
(F) 4 weeks after step (E), administering the anti-beta amyloid antibody to the patient in an amount of 6 mg/kg of body weight of the patient; and
(G) in consecutive intervals of 4 weeks after step (F), administering the anti-beta amyloid antibody to the patient in an amount of 10 mg/kg of body weight of the patient.

In other words, Protocol A comprises administering a first dose of anti-beta amyloid antibody to the patient in an amount of 1 mg/kg of body weight of the patient, followed by a second dose in an amount of 1 mg/kg of body weight four weeks after the first dose. In four week intervals after the second dose, antibody doses 3 and 4 are administered to the patient in an amount of 3 mg/kg of body weight. In four week intervals after administration of dose 4, doses 5 and 6 of the antibody are administered to the patient in an amount of 6 mg/kg of body weight. And then, four weeks after administration of dose 6, antibody dose 7 is administered to the patient in an amount of 10 mg/kg of body weight.

In some instances, after dose 7 of Protocol A, 5, 6, 7, 8, 9, or 10 doses of the anti-beta amyloid antibody in an amount of 10 mg/kg of body weight are administered to the patient. In certain instances, at least 10, at least 11, at least 12, at least 13, or at least 14 doses of the anti-beta amyloid antibody in an amount of 10 mg/kg of body weight of the subject are administered to the patient. In certain instances, at least 15, at least 16, at least 17, at least 18, at least 19, or at least 20 doses of the anti-beta amyloid antibody in an amount of 10 mg/kg of body weight of the subject are administered to the patient. In certain instances, 15 to 16, 15 to 17, 15 to 18, 15 to 19, 15 to 20, 15 to 21, 15 to 22, 15 to 23, 15 to 24, or 15 to 25 doses of the anti-beta amyloid antibody in an amount of 10 mg/kg of body weight of the subject are administered to the patient. In certain instances, the doses mentioned above are administered in consecutive intervals of 4 weeks. In certain instances, the doses mentioned above are administered to the patient intravenously.

In some instances, after dose 7 of Protocol A, at least 10 doses of the anti-beta amyloid antibody in an amount of 10 mg/kg of body weight of the subject are administered (e.g., intravenously) to the patient in uninterrupted 4 week intervals.

Another exemplary protocol according to the disclosure, designated Protocol B, comprises:
(a) administering the anti-Abeta-amyloid antibody to the subject in an amount of 1 mg/kg of body weight of the subject;
(b) 4 weeks after step (a), administering the anti-Abeta-amyloid antibody to the subject in an amount of 3 mg/kg of body weight of the subject;
(c) 4 weeks after step (b), administering the anti-Abeta-amyloid antibody to the subject in an amount of 6 mg/kg of body weight of the subject; and
(d) in consecutive intervals of 4 weeks after step (c), administering at least 10 doses of the anti-Abeta-amyloid antibody in an amount of 10 mg/kg of body weight of the subject.

In some instances, after step (d) of Protocol B, additional doses of the anti-beta amyloid antibody in an amount of 10 mg/kg of body weight are administered to the patient. In certain instances, at least 11, at least 12, at least 13, at least 14, at least 15, at least 16, at least 17, at least 18, at least 19, or at least 20 doses of the anti-beta amyloid antibody in an amount of 10 mg/kg of body weight of the subject are administered to the patient. In certain instances, at least 21, at least 22, at least 23, at least 24, at least 24, or at least 25 doses of the anti-beta amyloid antibody in an amount of 10 mg/kg of body weight of the subject are administered to the patient. In certain instances, 11 to 12, 11 to 13, 11 to 14, 11 to 15, 11 to 16, 11 to 17, 11 to 18, 11 to 19, 11 to 20, or 11 to 25 doses of the anti-beta amyloid antibody in an amount of 10 mg/kg of body weight of the subject are administered to the patient. In certain instances, the additional doses mentioned above are administered in consecutive intervals of 4 weeks. In certain instances, the doses mentioned above are administered to the patient intravenously.

In certain instances, when the patient develops an Amyloid Related Imaging Abnormality (ARIA)—e.g., ARIA-E, during the course of treatment is suspended until the ARIA resolves. In some instances, the treatment is suspended for 1 to 15 (1, 2, 3, 4, 5, 6, 7, 8, 9, 10, 11, 12, 13, 14, 15) weeks for the ARIA to resolve and then restarted. In certain cases, if the subject develops ARIA-E and/or ARIA-H accompanied by serious clinical symptoms, or ARIA-H with greater than or equal to 10 microhemorrhages and/or greater than or equal to two focal areas of superficial siderosis, or any new incident macrohemorrhage, treatment is permanently discontinued.

In certain instances, when the patient develops an Amyloid Related Imaging Abnormality (ARIA) during the course of treatment under Protocol A or B, the patient continues to be administered the doses described above without reduction of the dosage. In some instances, the dosage may be administered after the ARIA resolves.

If for some reason there is an interruption in the treatment (e.g., missed visits to the doctor or doctor recommendations due to ARIA or other side effects), the patient upon resumption of the treatment should continue at the same or higher dose. For example if the patient has already received two 3 mg/kg doses of the anti-beta amyloid antibody before interruption, upon resumption of treatment, the patient should be administered the 6 mg/kg dose. If the patient has already received two 6 mg/kg doses of the anti-beta amyloid antibody before interruption, upon resumption of treatment, the patient should be administered the 10 mg/kg dose. If the patient has already received two 10 mg/kg doses of the anti-beta amyloid antibody before interruption, upon resumption of treatment, the patient should be administered a 10 mg/kg dose and continue to be administered the 10 mg/kg dose for as long as possible.

The disclosure also features methods and compositions for treating mild Alzheimer's disease, early Alzheimer's disease, prodromal Alzheimer's disease, mild Alzheimer's disease dementia, or mild cognitive impairment due to Alzheimer's disease in a human subject in need thereof. The method involves administering to the human subject multiple doses of an anti-Abeta-amyloid antibody, wherein the method comprises administering in consecutive intervals of 4 weeks at least 6 doses of the antibody, wherein each dose is in an amount of 10 mg/kg of body weight of the subject. In some instances, the method comprises administering in consecutive intervals of 4 weeks at least 7 doses of the antibody, wherein each dose is in an amount of 10 mg/kg of body weight of the subject. In some instances, the method comprises administering in consecutive intervals of 4 weeks at least 8 doses of the antibody, wherein each dose is in an amount of 10 mg/kg of body weight of the subject. In some instances, the method comprises administering in consecutive intervals of 4 weeks at least 9 doses of the antibody, wherein each dose is in an amount of 10 mg/kg of body weight of the subject. In some instances, the method comprises administering in consecutive intervals of 4 weeks at least 10 doses of the antibody, wherein each dose is in an amount of 10 mg/kg of body weight of the subject. In some instances, the method comprises administering in consecutive intervals of 4 weeks at least 11 doses of the antibody, wherein each dose is in an amount of 10 mg/kg of body weight of the subject. In some instances, the method comprises administering in consecutive intervals of 4 weeks at least 12 doses of the antibody, wherein each dose is in an amount of 10 mg/kg of body weight of the subject. In some instances, the method comprises administering in consecutive intervals of 4 weeks at least 13 doses of the antibody, wherein each dose is in an amount of 10 mg/kg of body weight of the subject. In some instances, the method comprises administering in consecutive intervals of 4 weeks at least 14 doses of the antibody, wherein each dose is in an amount of 10 mg/kg of body weight of the subject. In some instances, the method comprises administering in consecutive intervals of 4 weeks at least 15 doses of the antibody, wherein each dose is in an amount of 10 mg/kg of body weight of the subject. In certain instances, all of the doses specified are administered without interruption even if the human subject develops an ARIA during the course of treatment. In certain instances, even if the doses are interrupted due to ARIA or other side effects, the treatment is continued at the same or higher dose of the antibody. If the patient was at the highest dose of Protocol A (10 mg/kg), upon resuming treatment after an interruption, the patient is to continue to be administered a 10 mg/kg dose of the antibody.

In some instances, the anti-beta amyloid antibody of the protocols and methods above comprises a VH and VL comprising the six CDRs of BIIB037. In certain instances, the anti-beta amyloid antibody comprises the VH and VL of BIIB037. In other instances, the anti-beta amyloid antibody comprises the heavy and light chains of BIIB037. In some instances, the anti-Abeta-amyloid antibody comprises a heavy chain variable region (VH) and a light chain variable region (VL), wherein the VH comprises a complementarity determining region (VHCDR1) with the amino acid sequence of SEQ ID NO:3, a VHCDR2 with the amino acid sequence of SEQ ID NO: 4, and a VHCDR3 with the amino acid sequence of SEQ ID NO:5, and wherein the VL comprises a VLCDR1 with the amino acid sequence of SEQ ID NO:6, a VLCDR2 with the amino acid sequence of SEQ ID NO:7, and a VLCDR3 with the amino acid sequence of SEQ ID NO: 8. In some instances, the anti-beta amyloid antibody comprises a VH comprising or consisting of SEQ ID NO: 1; and a VL comprising or consisting of SEQ ID NO:2. In some cases, the anti-Abeta-amyloid antibody comprises a heavy chain and a light chain, wherein: the heavy chain comprises or consists of SEQ ID NO:10; and the light chain comprises or consists of SEQ ID NO: 11.

Plasma p-tau

Plasma levels of phosphorylated tau (p-tau) are increased in Aβ positive human patients with mild cognitive impairment and further increased in Aβ positive human patients with AD. Clinical progression of AD is associated with changes in a number of different biomarkers prior to detection of measures of atrophy by MRI or changes in global cognition and ADL function. For example, Aβ42 (CSF and plasma) levels are among the first detectable changes, associated with cortical Aβ pathology, followed by changes in amyloid detected by PET, and then by changes in p-tau in CSF and plasma. See Hansson O. Nat Med. 2021; 27:954-963, incorporated herein by reference in its entirety.

Without wishing to be bound by theory, it is suggested that soluble p-tau may mediate the relationship between amyloid aggregates and tau aggregates. For example, increased levels of amyloid aggregates may lead to increased levels of soluble p-tau, which may in turn lead to increased tau tangle density. See, Mattsson-Carlgren N, et al. EMBO Mol Med. 2021; 13: e14022 and Mattsson-Carlgren N, et al. Sci Adv. 2020; 6: eaaz2387. Together, this suggests that amyloid-induced tau aggregation and spread (and consequent cognitive decline) might be driven by increases in soluble p-tau levels.

Conversely, the present disclosure encompasses a recognition that a reduction in amyloid aggregates may result in reduced p-tau levels, which could slow of accumulation of tau aggregates (and consequent clinical decline).

Various methods of the present disclosure include a step of determining a p-tau level in plasma of a human subject. Tau can be phosphorylated at a variety of different positions. In some embodiments, p-tau is phosphorylated at one or more positions selected from: Y18, S46, T50, S69, T71, S113, T123, T153, T175, T181, S184, S85, S191, Y197, S198, S199, S202, T205, S208, S210, T212, S214, T217, T231, S235, S237, S238, S258, S262, S289, S356, Y397, S400, T403, S404, S409, S412, S413, T414/S416, S422, T427, S433, and S435.

In some embodiments, a p-tau is or includes a phosphorylation at position 181, also referred to herein as p-tau¹⁸¹. In some embodiments, a p-tau is or includes a phosphorylation at position 217, also referred to herein as p-tau²¹⁷.

In some embodiments, a p-tau level in plasma of a human subject is determined at one or more time points.

In some embodiments, a p-tau level in plasma of a human subject is determined at a first and second time point, where the first time point is a baseline or reference measurement, and the second time point occurs after administering one or more doses of anti-beta-amyloid antibody. In some embodiments, the first time point is a baseline at time point that occurs before administering anti-beta-amyloid antibody. In some embodiments, a p-tau level in plasma at a first time point is a baseline that is adjusted. In some embodiments, a p-tau level in plasma at a first time point is a baseline that is unadjusted.

In some embodiments, a p-tau level in plasma at a second time point is reduced at least 5%, at least 6%, at least 7%, at least 8%, at least 9%, at least 10%, at least 11%, at least 12%, at least 13%, at least 14%, or at least 15% relative to a first time point (e.g., baseline). In some embodiments, a p-tau level in plasma at a second time point is reduced at least 10% relative to the first time point (e.g., baseline).

In some embodiments, a p-tau level in plasma of a human subject is compared to a reference measurement of p-tau in plasma. In some embodiments, a reference measurement of p-tau in plasma is, e.g., from healthy human subjects or human subjects diagnosed with AD that have not received treatment.

In some embodiments, a p-tau level in plasma of a human subject is compared to a reference level of p-tau of subjects who have been administered placebo. In some embodiments, a p-tau level in plasma of a human subject is reduced least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 24%, or at least 25% relative to p-tau levels of a placebo group of patients at the same time point.

### Methods

In some embodiments, the present disclosure provides methods of treating, monitoring, and/or evaluating a disease associated with the accumulation and deposition of beta amyloid, such as Alzheimer's disease, Down's syndrome, mild cognitive impairment, cerebral amyloid angiopathy, vascular dementia, and/or multi-infarct dementia.

In some embodiments, provided methods include methods of treating a human subject. In some embodiments, provided methods include methods of monitoring treatment response in a human subject. In some embodiments, provided methods include methods of determining a treatment plan for a human subject. In some embodiments, provided methods include methods of monitoring the cognitive state of a human subject.

In some embodiments, the present disclosure provides methods for selecting a patient for treatment with an anti-beta-amyloid antibody comprising determining a p-tau level in plasma of a human subject. In some embodiments, the present disclosure provides methods for determining a dose of an anti-beta-amyloid antibody to be administered to a human subject comprising determining a p-tau level in plasma of a human subject. In some embodiments, the present disclosure provides methods for selecting a patient receiving an anti-beta-amyloid antibody for cognitive support services comprising determining a p-tau level in plasma of a human subject.

In some embodiments, provided methods include methods of characterizing or assessing efficacy of an anti-beta-amyloid antibody in a human subject comprising determining a p-tau level in plasma of the human subject.

In some embodiments, the present disclosure provides methods of monitoring treatment response in a human subject having or suspected of having a disease associated with the accumulation and deposition of beta amyloid, such as Alzheimer's disease, Down's syndrome, mild cognitive impairment, cerebral amyloid angiopathy, vascular dementia, and/or multi-infarct dementia comprising determining a p-tau level in plasma of a human subject.

In some embodiments, the present disclosure provides methods of reducing plasma p-tau in a human subject having or suspected of having a disease associated with the accumulation and deposition of beta amyloid, such as Alzheimer's disease, Down's syndrome, mild cognitive impairment, cerebral amyloid angiopathy, vascular dementia, and/or multi-infarct dementia.

In some embodiments, provided methods comprise administering to a human subject an effective amount of an anti-beta-amyloid antibody. The route of administration of an anti-beta-amyloid antibody of the present disclosure may be by any suitable route, including intravenous, subcutaneous. In some embodiments, an anti-beta-amyloid antibody, described herein is systemically administered to a human subject via infusion. In some embodiments, an anti-beta-amyloid antibody is administered to a human subject by subcutaneous injection. In some embodiments, an anti-beta-amyloid antibody is administered to the human subject by intravenous infusion.

In some embodiments, the present disclosure provides methods that include administering an effective amount of an anti-beta-amyloid antibody to a human subject, where a level of plasma p-tau level has been or will be determined in the human subject.

In some embodiments, an anti-beta-amyloid antibody is administered to a subject in one or more doses. In some embodiments, an anti-beta-amyloid antibody is administered to a subject as a multidose regimen.

### Compositions

In some embodiments, the present disclosure provides compositions for use in treating a human subject with a disease associated with the accumulation and deposition of beta amyloid, such as Alzheimer's disease, Down's syndrome, mild cognitive impairment, cerebral amyloid angiopathy, vascular dementia, and/or multi-infarct dementia.

In some embodiments, a human subject has one or more characteristics. In some embodiments, a human subject is an ApoE carrier. In some embodiments, a human subject that has Down's syndrome. In some embodiments, a human subject is aged ≥65 years. In some embodiments, a human subject is aged 65 to 74 years. In some embodiments, a human subject is aged ≥75 years.

In some embodiments, the present disclosure provides compositions for use in treating a human subject with Alzheimer's disease, wherein a level of plasma p-tau has been determined prior to the treating. In some embodiments, a human subject has a plasma p-tau level prior to administration that is at or above a reference or threshold level. In some embodiments, a p-tau level in plasma comprises p-tau¹⁸¹ and/or p-tau²¹⁷. In some embodiments, the p-tau level is a plasma p-tau¹⁸¹ level.

In some embodiments, the present disclosure provides compositions for use in treating a human subject with Alzheimer's disease, wherein a level of plasma p-tau has been determined at a first time point that occurs prior to the treating and at a second time point. In some embodiments, a second time point for determining a p-tau level is after the human subject has received at least one dose of the anti-beta-amyloid antibody. In some embodiments, a second time point for determining the level of plasma p-tau is after at least 10 doses of anti-beta-amyloid antibody have been administered to the human subject. In some embodiments, a second time point for determining the level of plasma p-tau is after at least 14 doses of anti-beta-amyloid antibody have been administered to the human subject. In some embodiments, a second time point for determining the level of plasma p-tau is after at least 19 doses of anti-beta-amyloid antibody have been administered to the human subject. In some embodiments, the present disclosure provides compositions for treating a human subject whose change in plasma p-tau level between the first and second time point meets or exceeds a reference or threshold level. In some embodiments, a p-tau level in plasma comprises p-tau¹⁸¹ and/or p-tau²¹⁷. In some embodiments, the p-tau level is a plasma p-tau¹⁸¹ level.

In some embodiments, provided compositions comprise an anti-beta-amyloid antibody at a concentration of 50 mg/ml to 250 mg/ml; methionine at a concentration of 5 mM to 150 mM; Arg·HCl at a concentration of 50 mM to 200 mM; histidine at a concentration of 10 mM to 30 mM; PS80 at a concentration of 0.01% to 0.1%; and sucrose at a concentration of 0 to 3%. In some embodiments, a composition further comprises a thiol-containing antioxidant at a concentration of 0.02 mM to 4 mM. In some embodiments, a composition has a pH of 5.2 to 6.2. In some embodiments, a composition has a pH of 5.2 to 6.0. In some embodiments, a composition has a pH of 5.3 to 5.7. In some embodiments, a composition has a pH of 5.5. In some instances, the thiol-containing antioxidant is selected from the group consisting of GSH, GSSG, the combination of GSH and GSSG, cystine, cysteine, and the combination of cysteine and cystine. In some instances, the thiol-containing antioxidant is GSH. In some instances, the thiol-containing antioxidant is GSSG. In some instances, the thiol-containing antioxidant is the combination of GSH and GSSG.

In some embodiments, a composition comprises an anti-beta-amyloid antibody at a concentration of 100 mg/ml; Arg·HCl at a concentration of 150 mM; methionine at a concentration of 10 mM; histidine at a concentration of 10 mM to 30 mM; PS80 at a concentration of 0.01% to 0.1%; and sucrose at a concentration of 0 to 3%.

In some embodiments, a composition comprises an anti-beta-amyloid antibody at a concentration of 100 mg/ml; Arg·HCl at a concentration of 150 mM; methionine at a concentration of 10 mM; histidine at a concentration of 20 mM; and PS80 at a concentration of 0.05%.

In some embodiments, a composition comprises an anti-beta-amyloid antibody at a concentration of 175 mg/ml; Arg·HCl at a concentration of 150 mM; methionine at a concentration of 10 mM; histidine at a concentration of 10 mM to 30 mM; PS80 at a concentration of 0.01% to 0.1%; and sucrose at a concentration of 0 to 3%. In some embodiments, a composition further comprises a thiol-containing antioxidant at a concentration of 0.02 mM to 4 mM. In some instances, the thiol-containing antioxidant is selected from the group consisting of GSH, GSSG, the combination of GSH and GSSG, cystine, cysteine, and the combination of cysteine and cystine. In some instances, the thiol-containing antioxidant is GSH. In some instances, the thiol-containing antioxidant is GSSG. In some instances, the thiol-containing antioxidant is the combination of GSH and GSSG.

In some embodiments, a composition comprises an anti-beta-amyloid antibody at a concentration of 175 mg/ml; Arg·HCl at a concentration of 150 mM; methionine at a concentration of 10 mM; histidine at a concentration of 20 mM; and PS80 at a concentration of 0.05%. In some embodiments, a composition further comprises a thiol-containing antioxidant at a concentration of 0.02 mM to 4 mM. In some instances, the thiol-containing antioxidant is selected from the group consisting of GSH, GSSG, the combination of GSH and GSSG, cystine, cysteine, and the combination of cysteine and cystine. In some instances, the thiol-containing antioxidant is GSH. In some instances, the thiol-containing antioxidant is GSSG. In some instances, the thiol-containing antioxidant is the combination of GSH and GSSG.

### Kits

Also provided by the present disclosure are kits comprising a composition comprising an anti-beta-amyloid antibody for administration to a human subject and instructions for use. In some embodiments, the instructions direct administration to a human subject that has one or more characteristics. In some embodiments, the instructions direct administration to a human subject that is an ApoE carrier. In some embodiments, the instructions direct administration to a human subject that has Down's syndrome. In some embodiments, the instructions direct administration to a human subject that is aged ≥65 years. In some embodiments, the instructions direct administration to a human subject that is aged 65 to 74 years. In some embodiments, the instructions direct administration to a human subject that is aged ≥75 years.

In some embodiments, the instructions direct administration to a human subject that has a plasma p-tau level is above a reference or threshold level. In some embodiments, a p-tau level in plasma comprises p-tau¹⁸¹ and/or p-tau²¹⁷. In some embodiments, the p-tau level is a plasma p-tau¹⁸¹ level.

In some embodiments, kits for use in accordance with the present disclosure may include, instructions for processing samples, performing tests on samples, instructions for interpreting the results, etc.

In some embodiments, the instructions to determine a p-tau level in plasma of a human subject at a first and second time point. In some embodiments, a first time point for determining a p-tau level is prior to the human subject receiving a dose of the anti-beta-amyloid antibody. In some embodiments, a second time point for determining a p-tau level is after the human subject has received at least one dose of the anti-beta-amyloid antibody. In some embodiments, a second time point for determining the level of plasma p-tau is after at least 10 doses of anti-beta-amyloid antibody have been administered to the human subject. In some embodiments, a second time point for determining the level of plasma p-tau is after at least 14 doses of anti-beta-amyloid antibody have been administered to the human subject. In some embodiments, a second time point for determining the level of plasma p-tau is after at least 19 doses of anti-beta-amyloid antibody have been administered to the human subject.

In some embodiments, the instructions direct continued administration to a human subject whose change in plasma p-tau level between the first and second time point meets or exceeds a reference or threshold level. In some embodiments, a p-tau level in plasma comprises p-tau¹⁸¹ and/or p-tau²¹⁷. In some embodiments, the p-tau level is a plasma p-tau¹⁸¹ level.

### EXAMPLES

The invention is further illustrated by the following non-limiting examples. These Examples are set forth to aid in the understanding of the invention but are not intended to, and should not be construed to, limit its scope in any way. The Examples do not include detailed descriptions of conventional methods that would be well-known to those of ordinary skill in the art (molecular cloning techniques, etc.). Unless indicated otherwise, parts are parts by weight, molecular weight is average molecular weight, and temperature is indicated in Celsius. One having ordinary skill in the art would understand that the order of steps are not necessarily absolute and can vary to achieve the same outcome in certain embodiments.

### Example 1: Overview of Phase 3 Studies

The efficacy and safety of aducanumab in subjects with mild cognitive impairment (MCI) due to Alzheimer's disease or mild Alzheimer's disease dementia was evaluated in two identically designed Phase 3 studies, Study 1 and Study 2. The studies were designed based on an understanding of the mechanism of action of aducanumab, the outcomes of earlier studies, and the current understanding of the disease process and the underlying pathology. Table 1 provides an overview of the study design.

TABLE 1
Overview of Phase 3 Study Design

| | | |
|---|---|---|
| Population | 1. | MCI due to AD (~75-80%) + mild AD (~25-20%) |
| | 2. | MMSE 24-30, CDR global score 0.5, Repeatable |
| | | Battery for the Assessment of Neuropsychological |
| | | Status (RBANS) delayed memory index ≤85 |
| | 3. | Amyloid positive (by Aβ PET) |
| | 4. | 50-85 years of age |
| | 5. | ≥6 years of education or work experience |
| | 6. | Use of stable Alzheimer's disease symptomatic |
| | | medication on entry allowed |

| | |
|---|---|
| Study | 18-month placebo-controlled period + up to 5 years |
| duration | long-term extension (LTE) period |
| Sample | ~N = 1605 per study (n = 535/group) |
| size | Subjects randomized to placebo:low dose:high dose: |
| | in a 1:1:1 ratio |
| | Randomization stratified by site and apolipoprotein (ApoE) |
| | ε4 status (carrier or non-carrier) |
| Doses | Two aducanumab dose levels (low and high). Differential |
| | dosing based on ApoE ε4 status. |

These Phase 3 studies recruited earlier-stage patients who were Aβ positive as assessed by Aβ PET scans (by visual read) and who fulfilled clinical criteria for either MCI due to Alzheimer's disease or mild Alzheimer's disease dementia (as defined by NIA-AA criteria). Enrollment was monitored such that approximately 80% of the Phase 3 study populations would include subjects with a baseline clinical stage of MCI due to Alzheimer's disease (per the investigator's clinical assessment). Subjects were also required to have a CDR global score of 0.5, an RBANS score of ≤85 (based upon the Delayed Memory Index score), and an MMSE score between 24 and 30 (inclusive), and they must have had at least 6 years education or work experience. Subjects were to be 50 to 85 years of age at screening. Subjects with medical or neurological conditions other than Alzheimer's disease that may have contributed to the participant's cognitive impairment were excluded. Participants were to be in good health except for Alzheimer's disease.

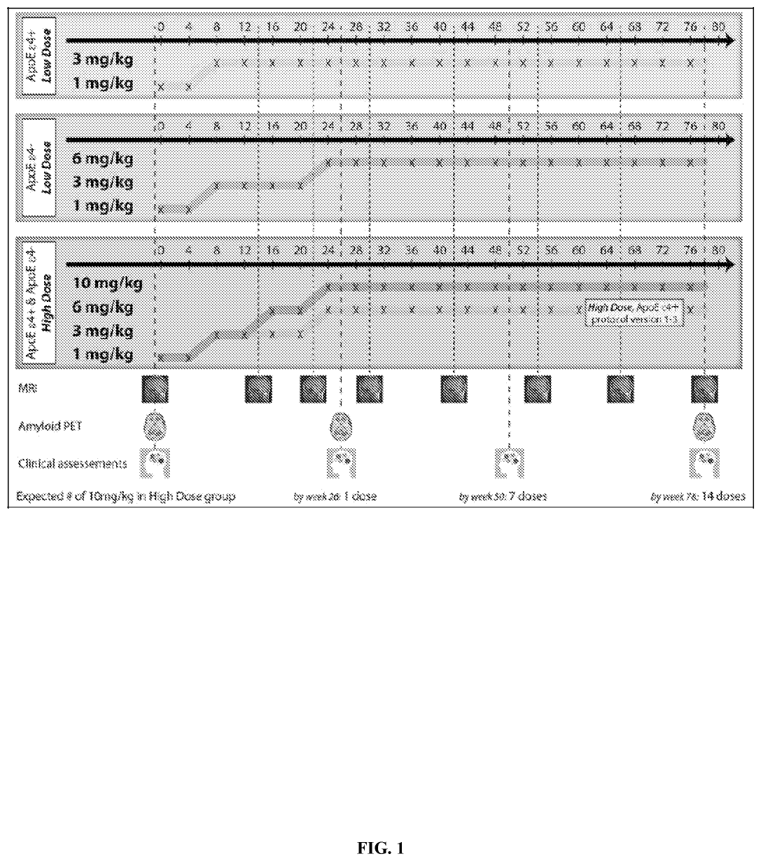

Of note, the Phase 3 protocols also required that subjects undergo ApoE genotyping, given the &4 allele is a major risk factor for Alzheimer's disease. Both ApoE &4 carriers and noncarriers were enrolled in both Phase 3 studies; however, differential dosing based on carrier status was limited to only the aducanumab “low” dose. See, FIG. 1.

### Example 2: Endpoints for Study 1

Primary endpoint results for the modified intent to treat (mITT) and Opportunity to Complete (OTC) subjects who had the opportunity to complete the Week 78 visit are summarized in Table 2.

In the high dose group, the advantage of aducanumab over placebo in mean change on the CDR-SB was −0.40 (23% less decline, nominal p=0.0101).

The low dose group also had less decline on the CDR-SB than placebo; however, the differences were smaller than in the high dose group and did not attain statistical significance.

TABLE 2
Change from Baseline in CDR-SB at Week 78: mITT and OTC Populations

| | mITT Population | | | OTC Population | | |
|---|---|---|---|---|---|---|
| | Diff vs PBO ^{a} | | | Diff vs PBO ^{a} | | |
| | p-value | | | p-value | | |
| | PBO decline | Low dose | High dose | PBO decline | Low dose | High dose |
| | (N = 548) | (N = 543) | (N = 547) | (N = 313) | (N = 329) | (N = 340) |
| CDR-SB | 1.74 | −0.25 | −0.40 | 1.61 | −0.26 | −0.37 |
| | | (−14%) | (−23%) | | (−16%) | (−23%) |
| | | 0.1171 | 0.0101 | | 0.1342 | 0.0309 |

^{a} difference vs placebo at Week 78. Negative percentage means less progression in the treated arm.
N: numbers of randomized and dosed subjects that were included in the analysis.
Data source: t-cdr-mmrm-pc-new/Output 6, t-cdr-mmrm-cplt-new/Output 7

Secondary efficacy endpoint results for the mITT dataset and the OTC dataset are summarized in Table 3.

For the high dose group, statistically significant differences from placebo (nominal p value<0.05) were observed for all secondary endpoints in both datasets except the MMSE in the mITT dataset. The low dose group did not show statistical significance on any of the 3 secondary endpoints in either the mITT or OTC datasets. However, a small, numeric advantage for low dose over placebo was observed on all endpoints except the MMSE.

TABLE 3
Change from Baseline in MMSE, ADAS-Cog13, and ADCS-
ADL-MCI at Week 78: mITT and OTC Populations

| | | | | OTC Population | | |
|---|---|---|---|---|---|---|
| | mITT Population | | | Diff vs PBO ^{a} | | |
| | Diff vs PBO ^{a} | | | (%) | | |
| | p-value | | | p-value | | |
| | PBO | Low | High | PBO | Low | |
| | decline | dose | dose | decline | dose | High dose |
| | (N = 548) | (N = 543) | (N = 547) | (N = 313) | (N = 329) | (=340) |
| MMSE | −33 | −0.1 | 0.5 | −3.0 | −0.1 | 0.7 |
| | | (3%) | (−159%) | | (3%) | (−23%) |
| | | 0.6900 | 0.0620 | | 0.8719 | 0.0324 |
| ADAS-Cog13 | 5.171 | 0.747 | −1.395 | 4.335 | −0.484 | −1.219 |
| | | (−14%) | (−27%) | | (−10%) | (−25%) |
| | | 0.1672 | 0.0098 | | 0.4103 | 0.0379 |
| ADCS-ADL- | −4.3 | 0.7 | 1.7 | −4.6 | 0.9 | 2.1 |
| MCI | | (−16%) | (−40%) | | (−20%) | (−46%) |
| | | 0.1556 | 0.0009 | | 0.1171 | 0.0002 |

^{a} difference vs placebo at Week 78. Negative percentage means less progression in the treated arm.
N: numbers of all randomized and dosed subjects that were included in the analysis.
Data source: t-mmse-mmrm-pc-new/Output 12, t-adas-mmrm-pc-new/Output 13, t-adl-mmrm-pc-new/Output 14, t-mmse-mmrm-cplt-new/Output 15, t-adas-mmrm-cplt-new/Output 16, t-adl-mmrm-cplt-new/Output 17

As a tertiary endpoint of Study 1, Brain Aβ was measured on PET and quantified as Standard Uptake Value Ratio (SUVR). Serial assessment of brain Aβ plaque levels as measured by Aβ PET and quantified as SUVR was conducted in the subset of subjects participating in the longitudinal Aβ PET sub-study. PET scans in the longitudinal sub-study were performed using the ¹⁸F-florbetapir Aβ PET tracer (except for a small number of subjects in whom another tracer was used). Results for the participants with ¹⁸F-florbetapir PET scans are summarized here.

For analyses of aducanumab's effect on brain Aβ plaque levels as measured by PET, a standard uptake value ratio (SUVR; the ratio of radiotracer uptake in regions expected to have Aβ pathology versus a reference region with minimal or no Aβ pathology) was calculated for a composite region of interest comprising the main cortical regions of the brain (parts of the frontal, parietal, lateral temporal, sensorimotor, anterior, and posterior cingulate) with whole cerebellum serving as the reference region [Ostrowitzki et al., Alzheimers Res. Ther. 8; 9 (1): 95 (2017); Chiao et al., J Nucl Med. 60 (1): 100-106 (2019); Sevigny, Nature 537 (7618): 50-6 (2016)]. This SUVR on the composite of regions was used as the primary endpoint for Aβ PET analysis. A negative change from baseline in the composite SUVR indicates a reduction in Aβ plaque level and a negative treatment difference (aducanumab minus placebo) favors aducanumab.

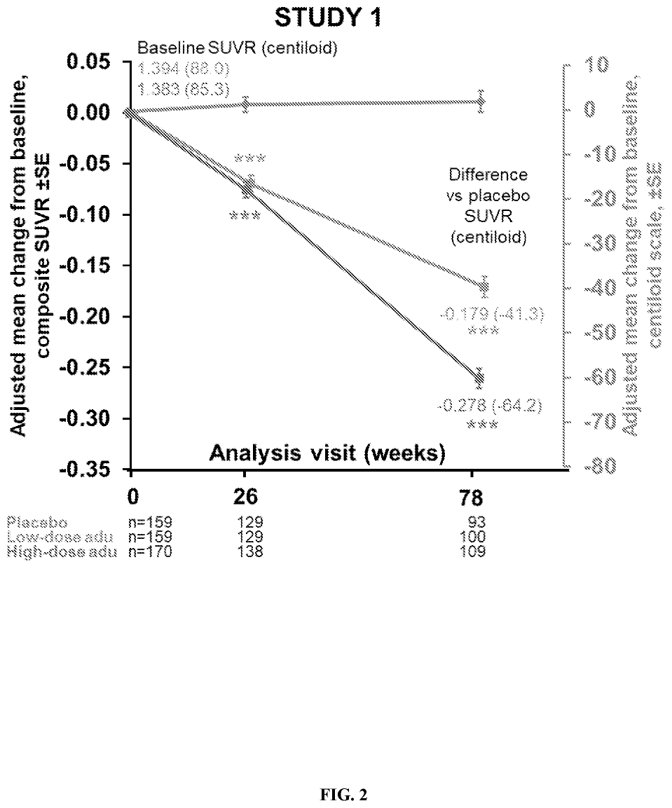

FIG. 2, depicts the time- and dose-dependent reductions in brain Aβ levels. At Week 26, coincident with the end of the titration phase, the adjusted mean change from baseline in Aβ PET composite SUVR was −0.070 and −0.076 in the low and high dose groups, respectively, compared with 0.007 in the placebo group. Due to the similarity of dosing during the titration phase, separation between the low and the high dose groups was not anticipated. At Week 78, the adjusted mean change from baseline in Aβ PET composite SUVR was −0.165 and −0.272 in the low and high dose groups, respectively, compared with 0.019 in the placebo group.

### Example 3: Endpoints of Study 2

Results of the mITT and OTC analyses of the primary endpoint show that aducanumab high dose did not reduce decline compared with placebo (Table 4). The low dose group did not show nominal statistical significance on the primary endpoint. However, a small, numeric advantage for the low dose over placebo was observed. This difference was similar in magnitude to the difference between low dose and placebo in Example 1.

TABLE 4
Change from Baseline in CDR-SB at Week 78:
mITT and OTC Populations, April Dataset

| | mITT Population | | | OTC Population | | |
|---|---|---|---|---|---|---|
| | Diff vs PBO ^{a} | | | Diff vs PBO ^{a} | | |
| | p-value | | | p-value | | |
| | PBO | Low | High | PBO | Low | High |
| | decline | dose | dose | decline | dose | dose |
| | (N = 545) | (N = 547) | (N = 555) | (N = 369) | (N = 370) | (N = 345) |
| CDR-SB | 1.55 | −0.18 | 0.03 | 1.45 | −0.11 | 0.08 |
| | | (−12%) | (2%) | | (−8%) | (6%) |
| | | 0.2362 | 0.8252 | | 0.4887 | 0.6266 |

^{a} difference vs placebo at Week 78. Negative percentage means less progression in the treated arm.
N: numbers of all randomized and dosed subjects that were included in the analysis.
Data source: t-cdr-mmrm-pc-new/Output 6, t-cdr-mmrm-cplt-new/Output 7

Results of the mITT and OTC analyses of the secondary endpoints show no statistically significant differences in decline on MMSE, ADAS-Cog13, or ADCS-ADL-MCI compared with placebo (Table 5). However, small numeric advantages for the low dose over placebo was observed on these endpoints. For ADAS-Cog13, or ADCS-ADL-MCI, results for the high dose group were similar to the low dose group.

TABLE 5
Change from Baseline in MMSE, ADAS-Cog13, and ADCS-ADL-MCI at
Week 78: Study 301, mITT and OTC Populations, April Dataset

| | mITT Population | | | OTC Population | | |
|---|---|---|---|---|---|---|
| | Diff vs PBO ^{a} | | | Diff vs PBO ^{a} | | |
| | (%) | | | (%) | | |
| | p-value | | | p-value | | |
| | PBO | Low | High | PBO | Low | High |
| | decline | dose | dose | decline | dose | dose |
| | (N = 545) | (N = 547) | (N = 555) | (N = 369) | (N = 370) | (N = 345) |
| MMSE | −3.5 | 0.2 | −0.1 | −3.2 | 0.1 | −0.4 |
| | | (−6%) | (3%) | | (−3%) | (13%) |
| | | 0.4875 | 0.7961 | | 0.7406 | 0.2367 |
| ADAS-Cog13 | 5.171 | −0.590 | −0.605 | 4.813 | −0.035 | −0.091 |
| | | (−11%) | (−12%) | | (−1%) | (−2%) |
| | | 0.2475 | 0.2446 | | 0.9495 | 0.8735 |
| ADCS-ADL- | −3.8 | 0.7 | 0.7 | −33 | 04 | 0.4 |
| MCI | | (−18%) | (−18%) | | (−12%) | (−12%) |
| | | 0.1345 | 0.1520 | | 0.4343 | 0.4051 |

^{a} difference vs placebo at Week 78. Negative percentage means less progression in the treated arm.
N: numbers of all randomized and dosed subjects that were included in the analysis.
Data source: t-mmse-mmrm-pc-new/Output 12, t-adas-mmrm-pc-new/Output 13, t-adl-mmrm-pc-new/Output 14, t-mmse-mmm-cplt-new/Output 15, t-adas-mmrm-cplt-new/Output 16, t-adl-mmrm-cplt-new/Output 17

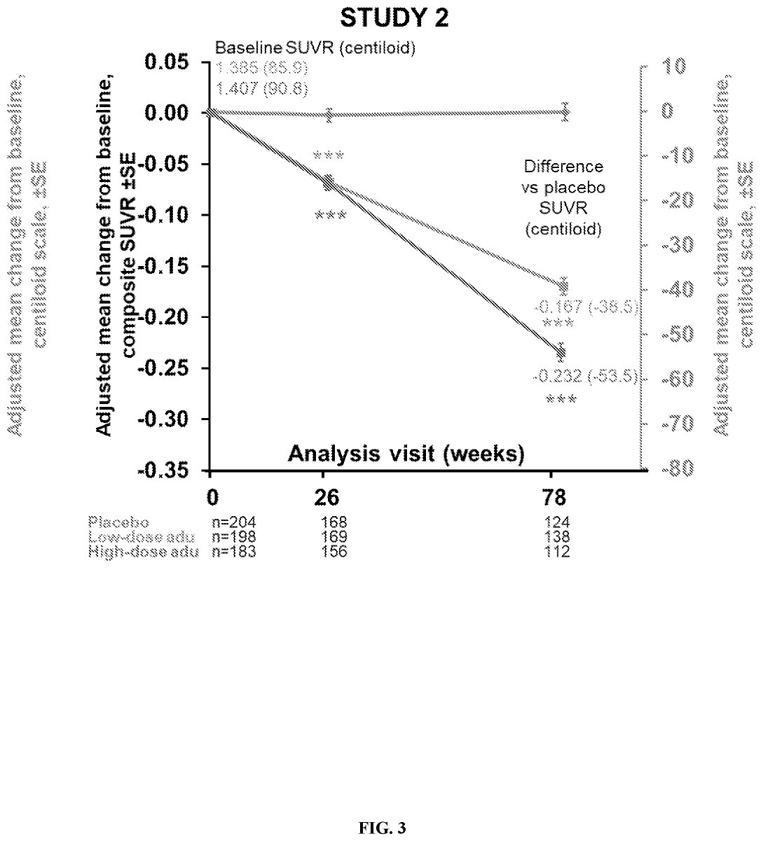

As a tertiary endpoint of Study 2, Brain Aβ was measured on PET and quantified as Standard Uptake Value Ratio (SUVR). As in the study of Example 2, a longitudinal Aβ PET sub-study was conducted using the ¹⁸F-florbetapir Aβ PET tracer. As can be seen in FIG. 3, aducanumab resulted in time- and dose-dependent reductions in brain Aβ levels. At Week 26, coincident with the end of the titration phase, the adjusted mean change from baseline in Aβ PET composite SUVR was −0.066 in both the aducanumab low and high dose groups, compared with −0.002 in the placebo group. Due to the similarity of dosing during the titration phase, separation between the low and the high dose groups was not anticipated. At Week 78, the adjusted mean change from baseline in Aβ PET composite SUVR was −0.168 and −0.238 in the aducanumab low and high dose groups, respectively, compared with −0.005 in the placebo group.

### Example 4: Plasma Levels of p-Tau from Study 1 and Study 2

The present example describes an analysis of effects of treatment with an exemplary anti-Aβ antibody, aducanumab, on plasma p-tau levels, specifically, plasma p-tau¹⁸¹ levels using data from the Phase 3 aducanumab trials described in Examples 1-3.

Participants with plasma samples at baseline and week 78 were assessed. A total of 6447 plasma samples from Study 1 and Study 2 subjects were analyzed using the Quanterix Simoa p-tau¹⁸¹ Advantage V2 kit at Frontage Laboratories' (Exton, PA) CLIA laboratory. A summary of the subjects from each study population is provided in Table 6 below.

TABLE 6
Patients from Phase 3 studies for Plasma pTau¹⁸¹ Analysis

| | Study 1 | Study 2 | Total |
|---|---|---|---|
| Plasma ptau¹⁸¹ analysis population | 885 | 953 | 1838 |

A more detailed description of these patients shows that baseline demographics and characteristics of Alzheimer's disease were similar across groups in the plasma p-tau¹⁸¹ analysis population, illustrated in Table 7.

TABLE 7
Baseline Demographics and Characteristics of Patients in Plasma pTau Analysis

| | STUDY 1 | | | | | | STUDY 2 | | | | | |
|---|---|---|---|---|---|---|---|---|---|---|---|---|
| | Placebo | | Low dose | | High dose | | Placebo | | Low dose | | High dose | |
| | (n = 294) | | (n = 297) | | (n = 294) | | (n = 334) | | (n = 334) | | (n = 285) | |
| Age in years, | 70.6 ± 7.32 | | 70.1 ± 7.51 | | 70.4 ± 7.36 | | 69.1 ± 7.76 | | 70.2 ± 6.99 | | 69.2 ± 7.93 | |
| mean ± SD | | | | | | | | | | | | |
| Female, n (%) | 154 | (52.4) | 137 | (46.1) | 149 | (50.7) | 171 | (51.2) | 177 | (53.0) | 151 | (53.0) |
| Race*, n (%) | | | | | | | 25 | (7.5) | | | | |
| Asian | 12 | (4.1) | 7 | (2.4) | 11 | (3.7) | | | 30 | (9.0) | 21 | (7.4) |
| Black or African | 0 | | 1 | (0.3) | 2 | (0.7) | 4 | (1.2) | 1 | (0.3) | 2 | (0.7) |
| American | | | | | | | | | | | | |
| White | 248 | (84.4) | 255 | (85.9) | 235 | (79.9) | 263 | (78.7) | 256 | (76.6) | 216 | (75.8) |
| Other or Not | 34 | (11.5) | 34 | (11.5) | 46 | (15.6) | 42 | (12.6) | 46 | (13.8) | 46 | (16.1) |
| Reported | | | | | | | | | | | | |
| Education | 14.7 ± 3.49 | | 14.7 ± 3.36 | | 14.7 ± 3.60 | | 15.0 ± 3.56 | | 14.7 ± 3.67 | | 15.0 ± 3.75 | |
| years, mean ± | | | | | | | | | | | | |
| SD | | | | | | | | | | | | |
| Alzheimer's | 158 | (53.7) | 161 | (54.2) | 158 | (53.7) | 185 | (55.4) | 200 | (59.9) | 170 | (59.6) |
| disease | | | | | | | | | | | | |
| medications | | | | | | | | | | | | |
| used, n (%) | | | | | | | | | | | | |
| ApoE ε4, n (%) | | | | | | | | | | | | |
| Carriers | 206 | (70.1) | 201 | (67.7) | 190 | (64.6) | 230 | (68.9) | 231 | (69.2) | 197 | (69.1) |
| Non-carriers | 88 | (29.9) | 96 | (32.3) | 104 | (35.4) | 103 | (30.8) | 103 | (30.8) | 88 | (30.9) |
| Clinical stage, | | | | | | | | | | | | |
| n (%) | | | | | | | | | | | | |
| MCI due to | 253 | (86.1) | 257 | (86.5) | 250 | (85.0) | 281 | (84.1) | 283 | (84.7) | 235 | (82.5) |
| Alzheimer's | | | | | | | | | | | | |
| disease | | | | | | | | | | | | |
| Mild | 41 | (13.9) | 40 | (13.5) | 44 | (15.0) | 53 | (15.9) | 51 | (15.3) | 50 | (17.5) |
| Alzheimer's | | | | | | | | | | | | |
| disease dementia | | | | | | | | | | | | |

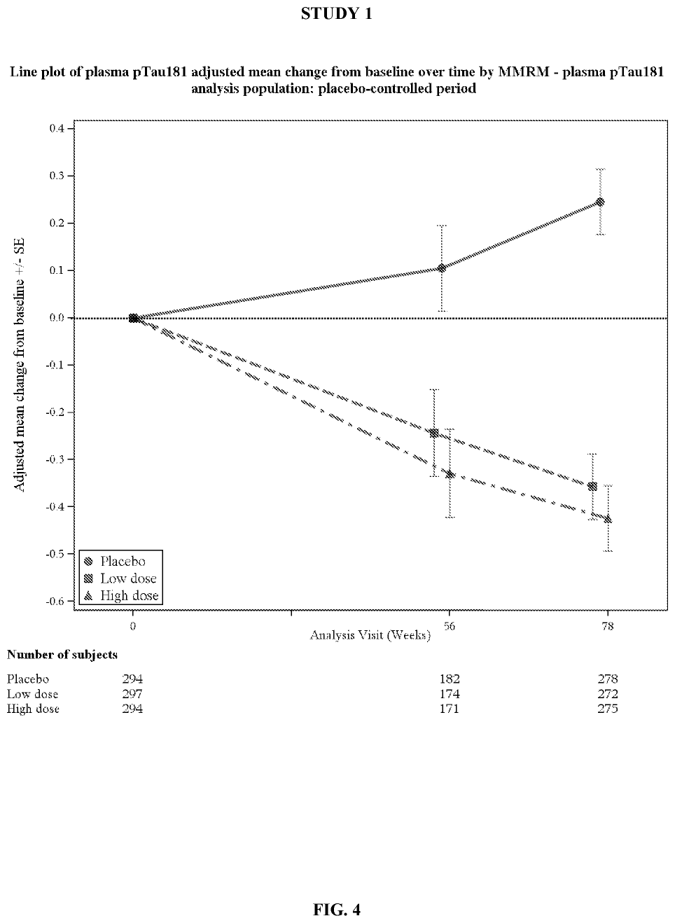

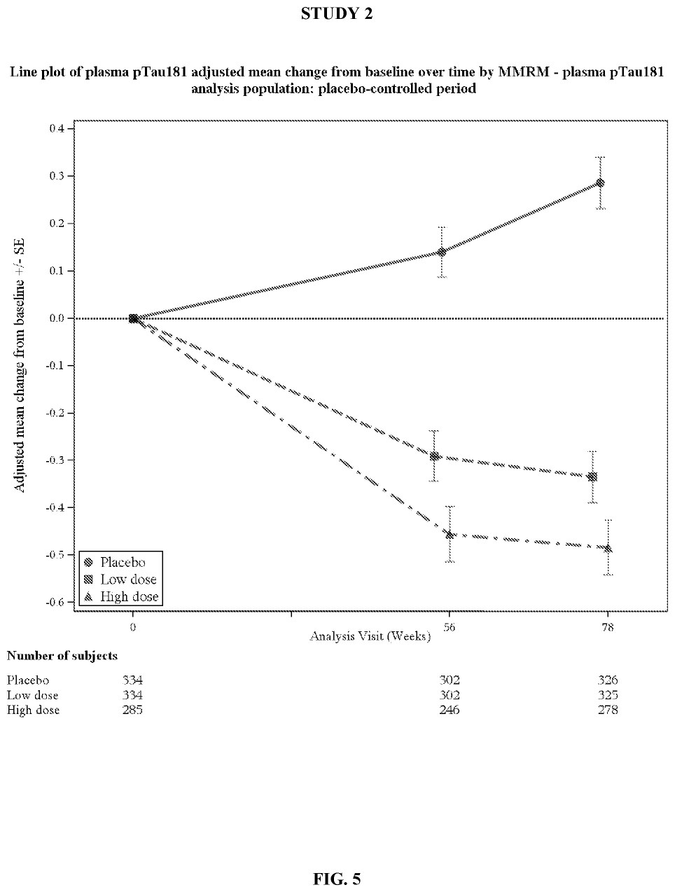

The adjusted mean change in in plasma p-tau¹⁸¹ levels were assessed for patients treated with placebo, low dose anti-Aβ antibody and high dose anti-Aβ antibody, and results are shown in FIG. 4 and FIG. 5 for the populations of Study 1 and Study 2, respectively. Mean baseline plasma p-tau¹⁸¹ levels between treatment groups and in Studies 1 and 2 were 3.264 pg/mL and 3.185 pg/mL, respectively; and median baseline levels were 2.980 pg/mL in Study 1 and 3.080 pg/mL in Study 2. In the placebo group, a time-dependent increase in plasma p-tau¹⁸¹ from baseline was seen in both Study 1 and Study 2 (8% and 9% increase from baseline, respectively). Mean change from baseline in plasma p-tau¹⁸¹ levels at week 78 in the high-dose aducanumab group vs. placebo was −0.424 (95% CI, −0.5607 to −0.2880; P<. 0001) for Study 1 and −0.484 vs. placebo (95% CI, −0.5978 to −0.3692; P<. 0001) for Study 2, reflecting a 13% and 15% decrease from baseline, respectively.

The effect of aducanumab on plasma p-tau¹⁸¹ levels also increased as the total cumulative dose increased. The mean cumulative dose received up to week 78 were 127 in Study 1 and 122 in Study 2. Subgroup analysis of subsets of high dose subjects defined by the total cumulative dose received up to week 78 was performed. Relative reduction versus placebo between aducanumab treatment groups in mean change from baseline in plasma p-tau¹⁸¹ was seen across all three cumulative dose groups (<100 mg/kg; 110-149 mg/kg, and ≥150 mg/kg). Also, a greater treatment effect on plasma p-tau¹⁸¹ levels with greater total cumulative dose (data not shown). For the highest total cumulative dose at week 78 (≥150 mg/kg), a reduction from baseline in plasma p-tau¹⁸¹, levels was observed in both Study 1 and Study 2 (17% and 19% reduction, respectively).

The present analysis revealed that treatment with an exemplary anti-Aβ antibody, aducanumab, significantly lowers plasma p-tau¹⁸¹ levels. Moreover, the effect of aducanumab on plasma p-tau¹⁸¹ levels was observed to be both time and dose dependent.

### Example 5: Plasma p-Tau is Correlated with Change in Amyloid PET SUVR

This example describes a relationship between an anti-beta-amyloid antibody induced change in plasma p-tau levels, here p-tau¹⁸¹, and brain amyloid levels as expressed as amyloid PET standard uptake value ratios (SUVR).

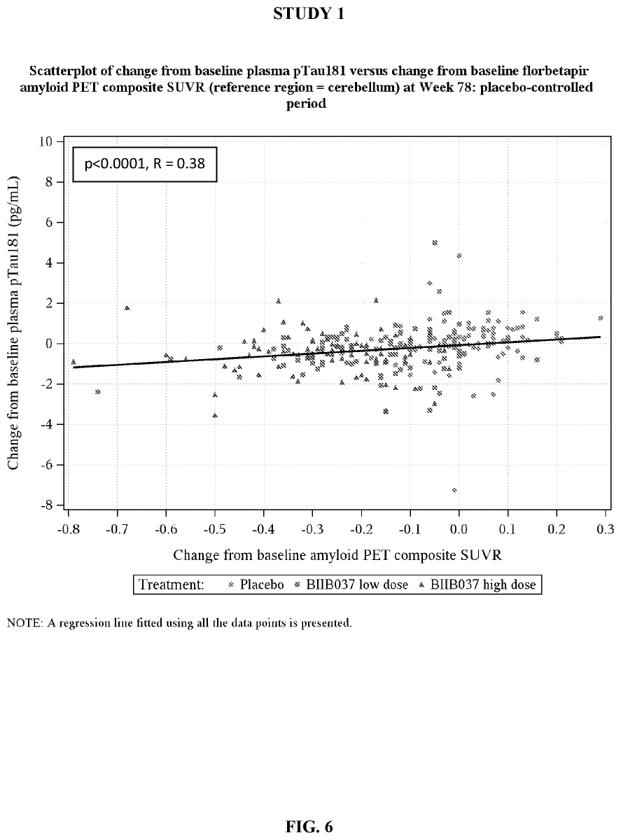

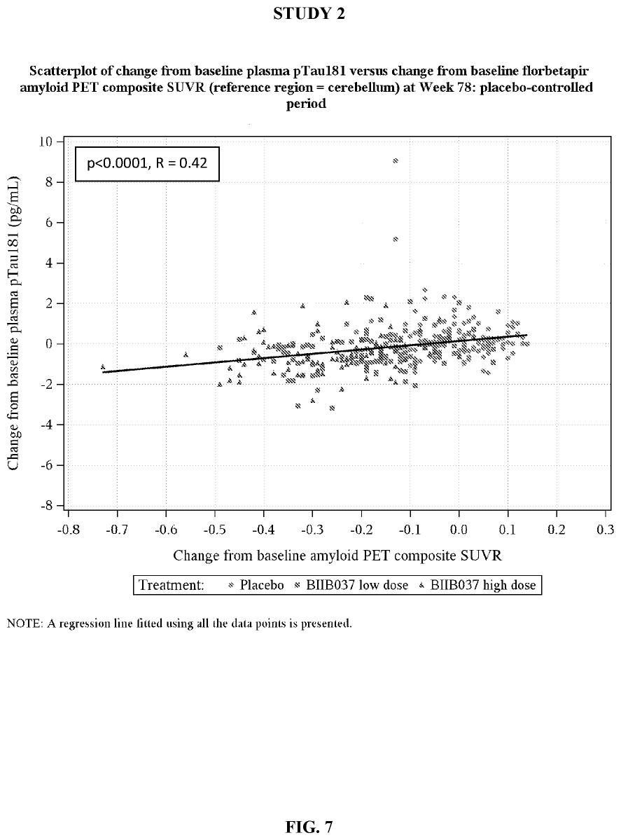

FIG. 6 and FIG. 7 depict scatterplots of change from baseline plasma p-tau¹⁸¹ vs change from baseline florbetapir amyloid PET composite SUVR (reference region=cerebellum) for the population of Study 1 and Study 2, respectively. Subjects were assessed at week 78, and subjects were separated into the following categories: those receiving placebo (circles), those subjects receiving “low dose” anti-Aβ antibody treatment (squares), and those subjects receiving “high dose” anti-Aβ antibody treatment (triangles). There was a dose dependent correlation of reduction in p-tau¹⁸¹ levels and reduction in amyloid deposits.

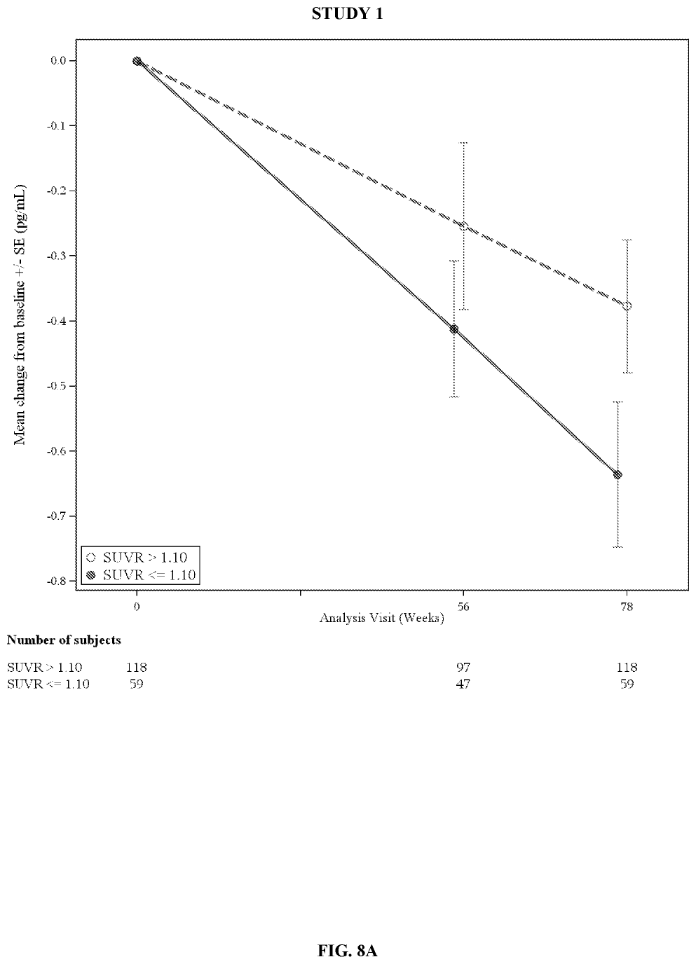

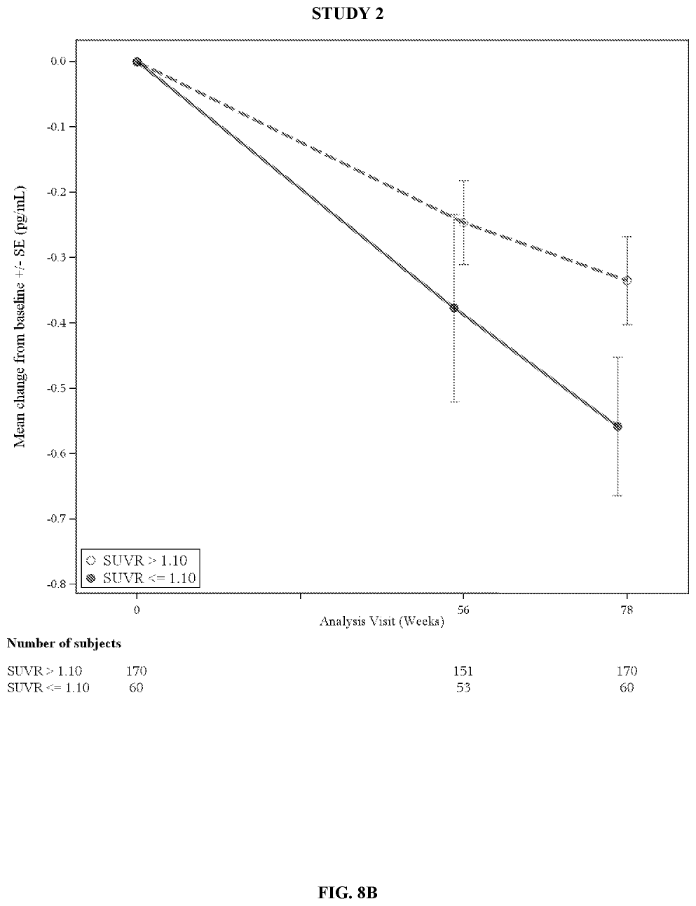

Moreover, those subjects who had a lower SUVR were observed to also have a greater decrease from baseline of plasma p-tau¹⁸¹. As depicted in FIG. 8A and FIG. 8B, there was a greater change from baseline plasma p-tau¹⁸¹ in subjects whose baseline florbetapir amyloid PET composite SUVR was ≤1.10 (closed circles, solid line) compared to those subjects whose baseline florbetapir amyloid PET composite SUVR was greater than >1.10 (open circles, dashed line), which was observed in both the Study 1 and Study 2 populations of patients.

This example demonstrates that the treatment effect of aducanumab on plasma p-tau¹⁸¹ was associated with lowering of amyloid PET SUVR.

### Example 6: Greater Reduction in Plasma p-Tau is Associated with Improved Clinical Measures

The present example describes a relationship between plasma p-tau levels and clinical outcomes. Specifically, a greater reduction in plasma p-tau, e.g., plasma p-tau¹⁸¹, was associated with less clinical decline by multiple measures of clinical efficacy. As shown in Table 8 below, correlations between plasma p-tau¹⁸¹ and clinical efficacy were observed across four different clinical measures in patients from both Study 1 and Study 2, and in the expected directions.

TABLE 8
Association between change in p-tau and clinical efficacy

| Association between | | | Correlation (p-value) | |
|---|---|---|---|---|
| change in p-tau and | | Expected | Study 1 | Study 2 |
| efficacy at Week 78 | | correlation | (n = 514-521) | (n = 577-581) |
| p-tau¹⁸¹ | CDR-SB | Positive | 0.11 | 0.15 |
| | | | (0.0137) | (0.0002) |
| | MMSE | Negative | −0.20 | −0.16 |
| | | | (<0.0001) | (0.0001) |
| | ADAS-Cog13 | Positive | 0.16 | 0.15 |
| | | | (0.0003) | (0.0002) |
| | ADCS-ADL-MCI | Negative | −0.12 | −0.13 |
| | | | (0.0056) | (0.0013) |

These findings demonstrate that modification of biomarkers fundamental to the underlying disease pathology (e.g., p-tau, e.g., p-tau¹⁸¹) was associated with statistically significant slowing of clinical decline as measured by CDR-SB, MMSE, ADAS-Cog13, and ADCS-ADL-MCI. Thus, the treatment effect of aducanumab on plasma p-tau¹⁸¹ was associated with reduced cognitive and functional decline.

Together, these examples highlight important new data from ˜7,000 plasma samples from the aducanumab Phase 3 trials that, for the first time, examines the effect of aducanumab on plasma phosphorylated tau¹⁸¹ (p-tau¹⁸¹) and its correlation to amyloid beta plaques and disease progression, as measured by clinical decline endpoints, in patients with early Alzheimer's disease. The accumulation of amyloid beta plaques and tangles of tau proteins in brain cells are the two defining pathologies of Alzheimer's disease; the present disclosure demonstrates a correlation between plasma p-tau reduction, amyloid beta plaque levels and clinical decline in Alzheimer's disease.

### Example 7: Subgroup Analysis of Plasma p-Tau

The present example describes analysis of the effects of treatment with an anti-beta-amyloid antibody, aducanumab, on plasma p-tau levels in different subgroups assessed at week 78. Specifically, the present example provides subgroup analysis of plasma p-tau¹⁸¹ levels in subjects of from Study 1 and Study 2, differentiated by age (≤64, 65 to 74, or ≥75 years), ApoE ε4 status (carrier or noncarrier), and baseline clinical stage (MCI due to AD or mild AD dementia).

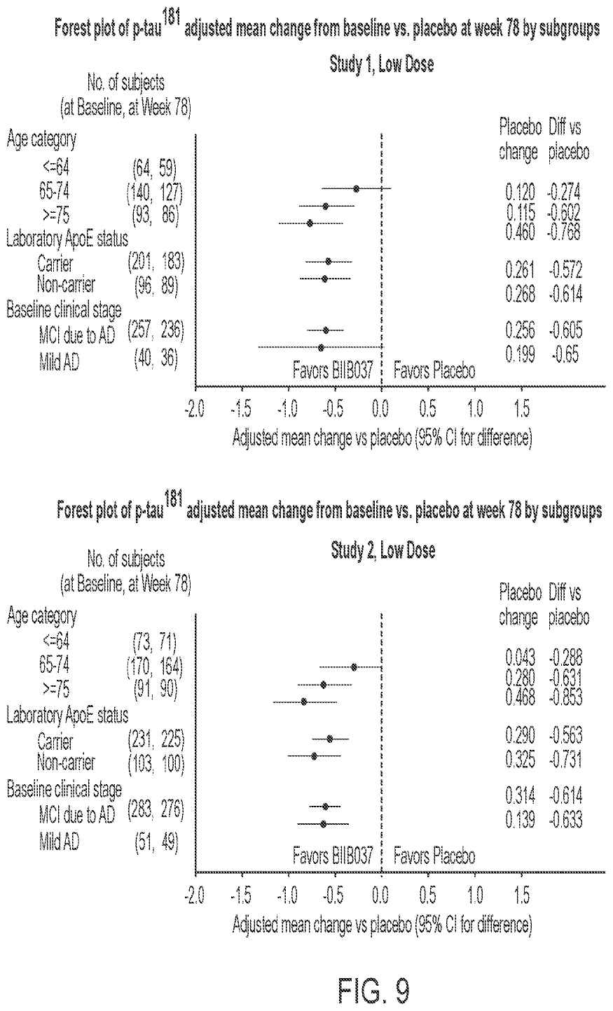

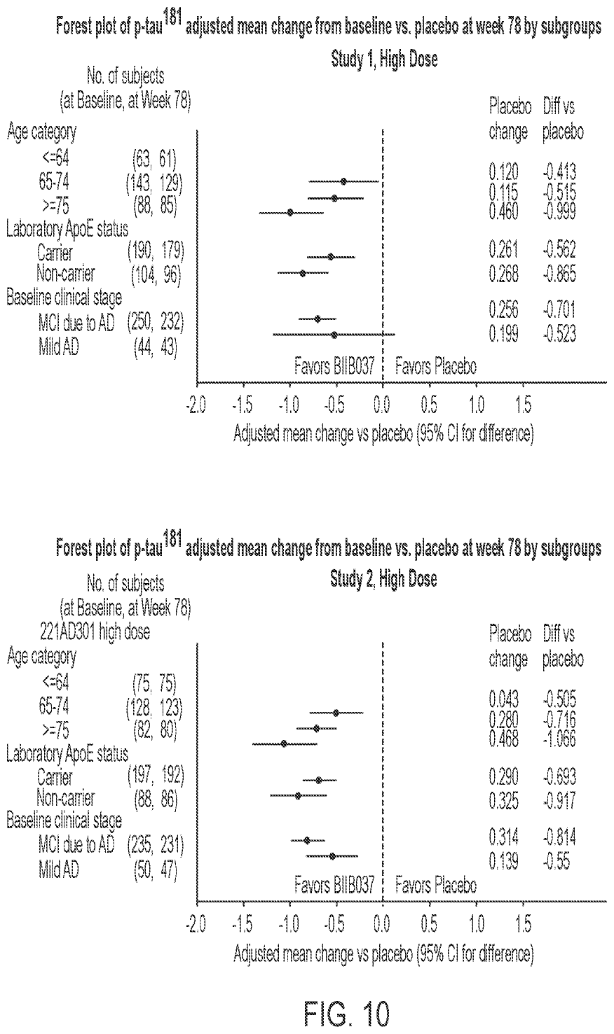

Results are depicted in FIG. 9 (for low dose treated patients) and FIG. 10 (for high dose treated patients). Results were based on an MMRM (mixed model for repeated measures) model for each subgroup, with change from baseline as a dependent variable. Subgroup analysis was also performed based on sex (male/female) and baseline AD medication use (data not shown).

A reduction in plasma p-tau¹⁸¹ levels was observed in both high-dose and low-dose aducanumab treated groups compared with placebo across subgroups in both Study 1 and Study 2. Results were consistent with overall plasma p-tau¹⁸¹ results from all subjects described above. Overall, subgroup analysis revealed a reduction in plasma p-tau¹⁸¹ levels in subjects treated with aducanumab for all subgroups investigated. These data support that aducanumab has a treatment effect across subjects.

A reduction in plasma p-tau¹⁸¹ was seen in both male and female subjects, and subjects stratified by AD symptomatic medication at baseline, in comparison with placebo. This reduction was seen across treatment groups (both low dose and high dose) in both studies at week 78, with no apparent trends between subjects stratified by sex or AD medication as baseline.

Moreover, it was consistently observed that there was a greater reduction in plasma p-tau 181 levels in individuals who were ≥75 years old at baseline and in ApoE &4 noncarriers. The reduction in plasma p-tau¹⁸¹ levels in subjects ≥75 years old is also reflected in Table 9 below, which provides percent relative change in plasma p-tau¹⁸¹ levels from baseline and from placebo. Similarly, Table 10 provides the percent relative change in plasma p-tau¹⁸¹ levels from baseline and from placebo for ApoE carriers and noncarriers.

TABLE 9
Percent relative change in plasma p-tau¹⁸¹ at Week 78 - by Age

| | Placebo from | | Low Dose | | High Dose | | Low Dose/ |
|---|---|---|---|---|---|---|---|
| | baseline | | from baseline | | from baseline | | High Dose |
| | (n=) | | (n=) | | (n=) | | from placebo |
| Age 64-74, Study 2 | +9% | (155) | −11% | (164) | −14% | (123) | −20%/−23% |
| Age ≥75, Study 2 | +15% | (87) | −11% | (90) | −18% | (80) | −26%/−33% |
| Age 64-74, Study 1 | +4% | (140) | −15% | (127) | −12% | (129) | −19%/−16% |
| Age ≥75, Study 1 | +15% | (86) | −10% | (86) | −16% | (85) | −25%/−31% |

TABLE 10
Percent relative change in plasma p-tau¹⁸¹ at Week 78 - by ApoE carrier status

| | Placebo from | | Low Dose | | High Dose | | Low Dose/ |
|---|---|---|---|---|---|---|---|
| | baseline | | from baseline | | from baseline | | High Dose |
| | (n=) | | (n=) | | (n=) | | from placebo |
| ApoE carrier, Study 2 | +9% | (227) | −8% | (225) | −13% | (192) | −17%/−22% |
| ApoE noncarrier, Study 2 | +10% | (99) | −12% | (100) | −19% | (86) | −22%/−29% |
| ApoE carrier, Study 1 | +8% | (193) | −10% | (183) | −9% | (179) | −18%/−17% |
| ApoE noncarrier, Study 1 | +9% | (85) | −10% | (89) | −19% | (96) | −19%/−28% |

As noted above, a reduction in plasma p-tau¹⁸¹ is associated with improved clinical measures. Thus, the present disclosure recognizes that certain subgroups (e.g., subjects ≥75 years at baseline and/or subjects that are ApoE &4 noncarriers) may have improved benefits from treatment with anti-beta-amyloid antibody (e.g., aducanumab).

In summary, the plasma p-tau¹⁸¹ subgroup analysis of the present example revealed a consistent reduction in plasma p-tau¹⁸¹ levels in all participants treated with aducanumab, with an even greater decrease in those patients ≥75 years old at baseline and in ApoE &4 noncarriers, which may be associated with improved benefits from treatment with aducanumab.

### Example 8: Subgroup Analysis Based on Baseline Amyloid and Baseline p-Tau Levels

The present example describes further subgroup analysis of the effects of treatment with an anti-beta-amyloid antibody, aducanumab, on plasma p-tau levels. Specifically, the present example provides subgroup analysis of plasma p-tau¹⁸¹ levels in subjects of from Study 1 and Study 2 at week 78, differentiated by baseline amyloid PET SUVR and baseline plasma p-tau¹⁸¹ levels.

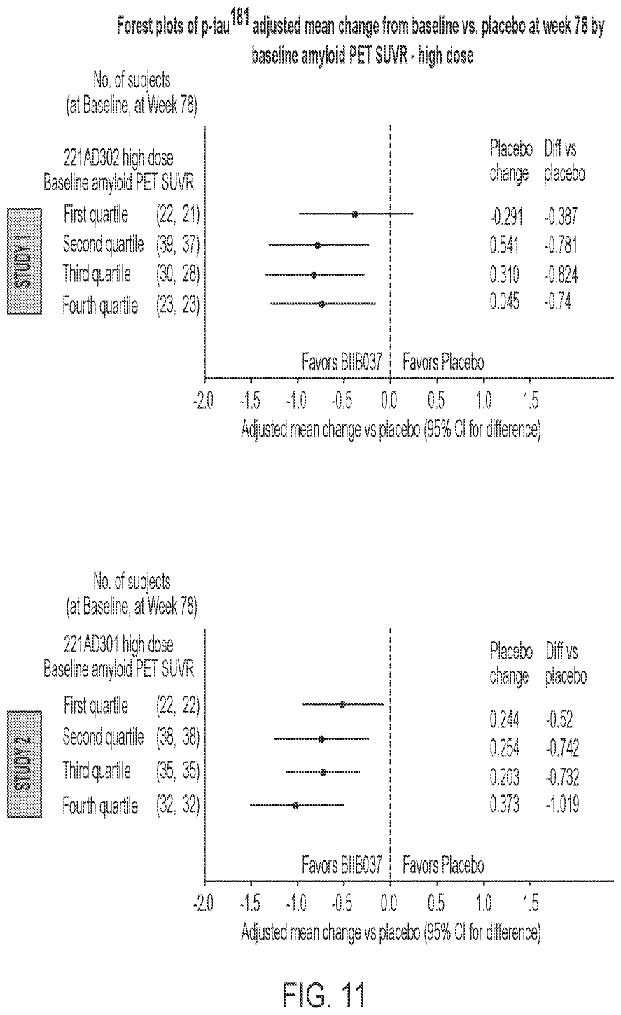

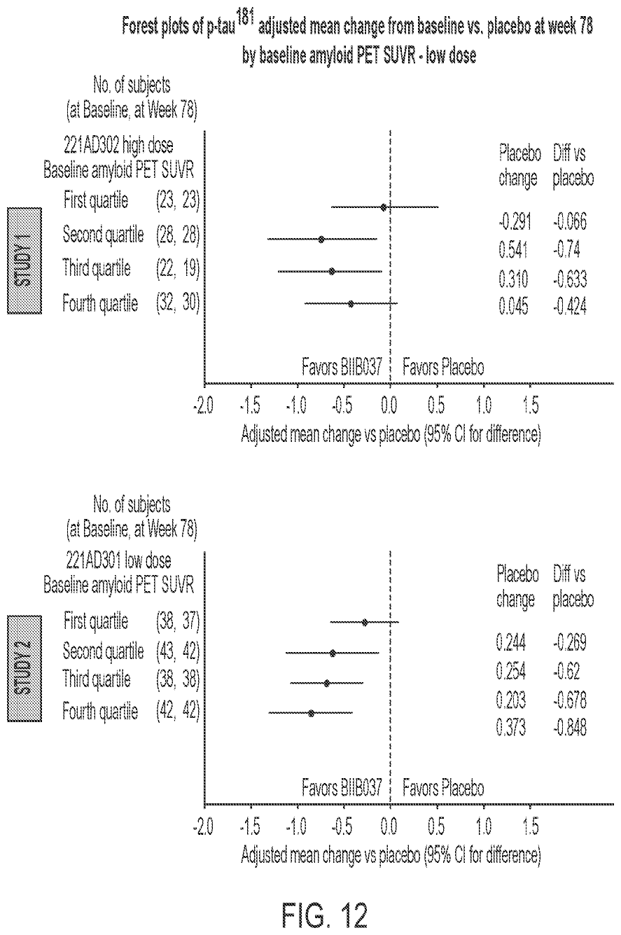

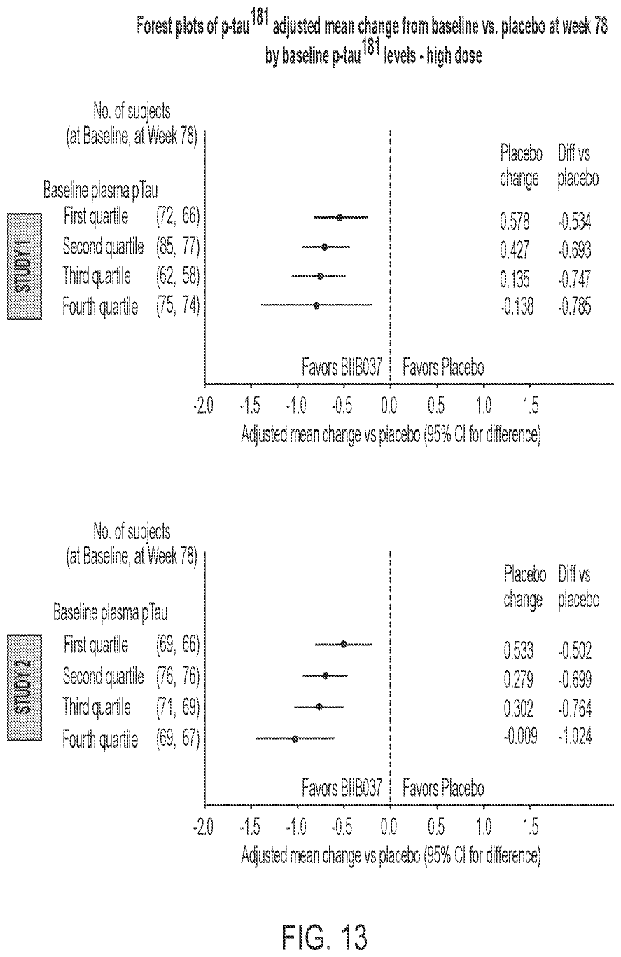

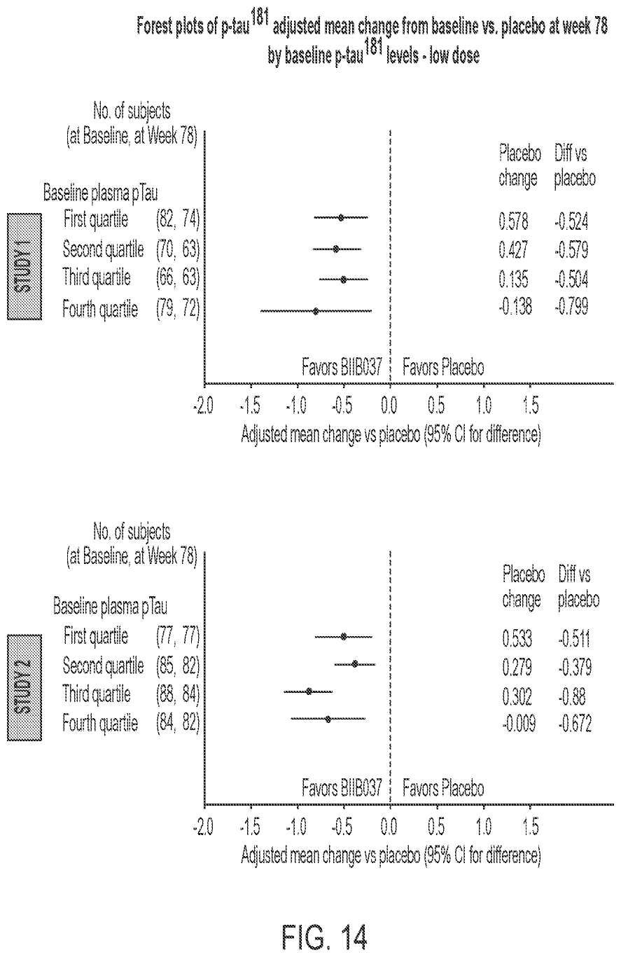

Subjects were stratified into quartiles of relative baseline levels. Results for subgroup analysis of baseline amyloid PET SUVR are depicted in FIG. 11 (for high dose treated patients) and FIG. 12 (for low dose treated patients). Results for subgroup analysis of baseline plasma p-tau¹⁸¹ levels are depicted in FIG. 13 (for high dose treated patients) and FIG. 14 (for low dose treated patients).

A reduction in plasma p-tau¹⁸¹ levels was consistently observed in both high-dose and low-dose aducanumab treated groups compared with placebo in both Study 1 and Study 2 across all four quartiles of baseline amyloid PET SUVR and also across all quartiles of baseline plasma p-tau¹⁸¹. These results support that aducanumab is an effective treatment for subjects at all baseline levels of PET SUVR and all baseline plasma p-tau¹⁸¹ levels.

Moreover, it was consistently observed that there was a greater reduction in plasma p-tau¹⁸¹ levels in the subgroup with the highest baseline plasma p-tau¹⁸¹ levels, namely those in the fourth quartile, as depicted in FIG. 13 and FIG. 14. As noted above, a reduction in plasma p-tau¹⁸¹ level is associated with clinical response. This data supports that subjects with a higher plasma p-tau¹⁸¹ level at baseline may show increased response to treatment with anti-beta-amyloid antibody (e.g., aducanumab).

### Example 9: Effect of Treatment on Plasma p-Tau Levels in Long-Term Extension Study

The present example describes analysis of the effect of treatment with an anti-beta-amyloid antibody, aducanumab, on plasma p-tau levels in a long-term extension study. The long term extension study includes the placebo controlled trial period plus a long-term extension (LTE) period. Subjects were randomized into “early start” and “late start” treatment groups. Early start subjects are those who at the start of the placebo-control period were randomized to aducanumab and continued their originally assigned treatment in the LTE period, and include “early start low-dose” and “early start high-dose” subjects. Late start subjects are those who at the start of the placebo-control period were randomized to placebo and then during the LTE period were randomized to receive dose-blinded aducanumab (low:high 1:1), and include “late start low-dose” and “late start high-dose” subjects.

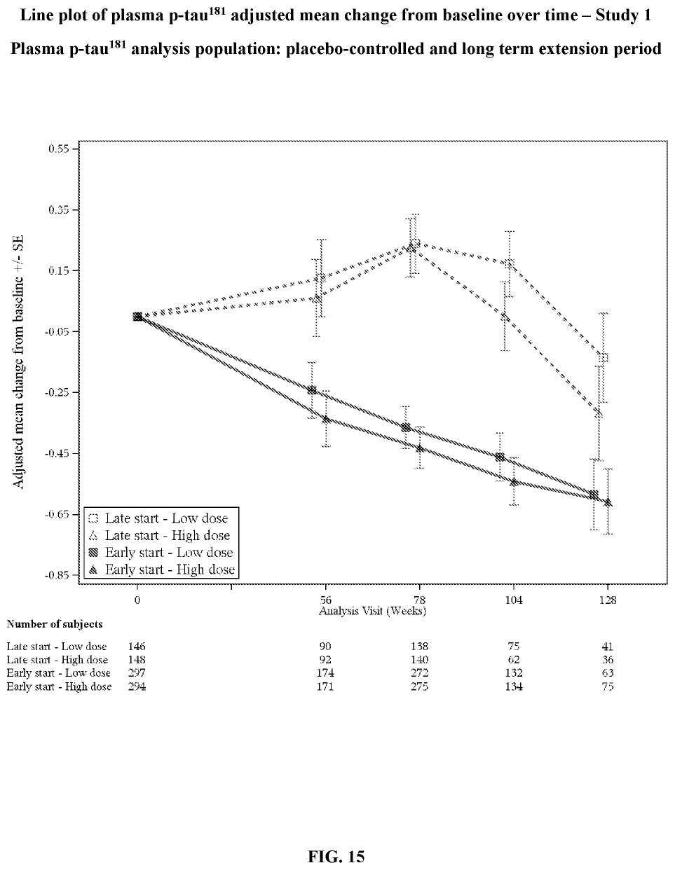

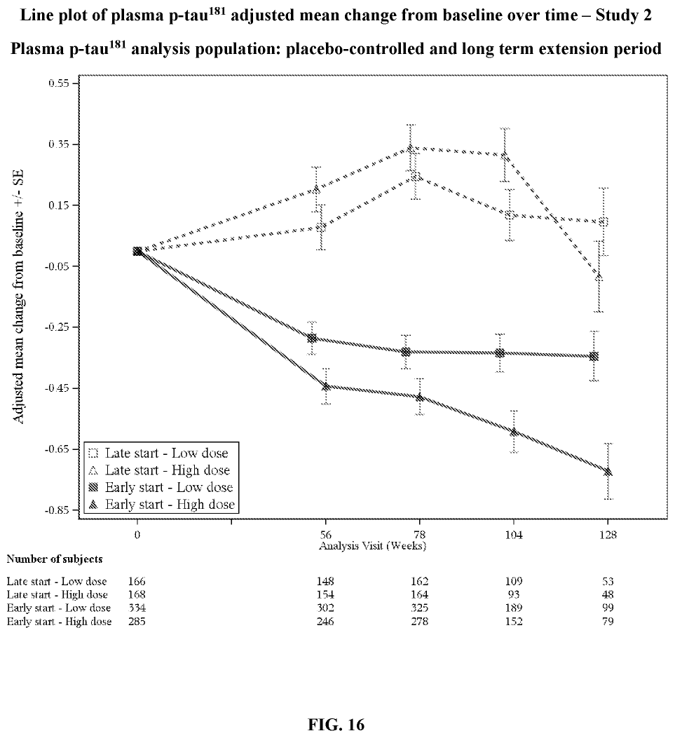

Results are depicted in FIG. 15 and FIG. 16. In both Study 1 and Study 2, plasma p-tau¹⁸¹ levels of late start subjects increased during the placebo-controlled period and started to reduce in the long-term extension period once subjects started to receive aducanumab treatment. At week 128, the late start high-dose group of subjects had a larger reduction in plasma p-tau¹⁸¹ levels than the late start low-dose group, aligning with the larger exposure in the long-term extension up to week 128.

The plasma p-tau¹⁸¹ levels of subjects in the early start groups continued to reduce in the long-term extension in both Study 1 and Study 2. Comparison of the late start and early start groups within each study revealed statistically significant differences at both week 104 and week 128. For example, change from baseline in plasma p-tau¹⁸¹ for the early start high-dose group compared to the late start high-dose group were and −0.541 (p<0.0001) and −0.908 (p<0.0001) at Week 104, and −0.289 (p=0.1265) and −0.640 (p<0.0001) for Study 1 and Study 2, respectively. A similar trend was observed for subjects out to week 134 (data not shown).

This example demonstrates that high dose of aducanumab was associated with a greater reduction in plasma p-tau levels and that longer duration of treatment continued to reduce plasma p-tau¹⁸¹ levels beyond 78 weeks.

### EXEMPLARY EMBODIMENTS

1. A method, comprising
determining a p-tau level in plasma of a human subject, and
administering one or more doses of an anti-beta-amyloid antibody to the human subject.

2. The method of embodiment 1, wherein the p-tau level in plasma of a human subject is determined prior to the human subject receiving a dose of the anti-beta-amyloid antibody.

3. The method of embodiment 1 or 2, wherein the method further comprises determining a p-tau level in plasma of a human subject at a first and second time point.

4. The method of embodiment 3, wherein the first time point is prior to the human subject receiving a dose of the anti-beta-amyloid antibody.

5. The method of embodiment 3 or 4, wherein the second time point is after the human subject has received at least one dose of the anti-beta-amyloid antibody.

6. The method of any one of embodiments 3-5, wherein the second time point for determining the level of plasma p-tau is after at least 10 doses of anti-beta-amyloid antibody have been administered to the human subject.

7. The method of any one of embodiments 3-6, wherein the second time point for determining the level of plasma p-tau is after at least 14 doses of anti-beta-amyloid antibody have been administered to the human subject.

8. The method of any one of embodiments 3-7, wherein the second time point for determining the level of plasma p-tau is after at least 19 doses of anti-beta-amyloid antibody have been administered to the human subject.

9. The method of any one of embodiments 5-8, wherein the first time point occurs before the second time point, and, if the level of plasma p-tau at a first time point is greater than the level of plasma p-tau at the second time point, the method further comprises a second administration of the anti-beta-amyloid antibody to the human subject.

10. The method of embodiment 9, wherein the second administration of the anti-beta-amyloid antibody includes a dose of the anti-beta-amyloid antibody that is equal to or greater than the dose of the anti-beta-amyloid antibody administered to the human subject in the first administration.

11. The method of any one of embodiments 5-8, wherein the first time point occurs before the second time point, and, if the level of plasma p-tau at a first time point is less than the level of plasma p-tau at the second time point, the method further comprises a second administration of the anti-beta-amyloid antibody to the human subject.

12. The method of embodiment 11, wherein the second administration of the anti-beta-amyloid antibody includes a dose of the anti-beta-amyloid antibody that is equal to or less than the dose of the anti-beta-amyloid antibody administered to the human subject in the first administration.

13. The method of any one of embodiments 1-12, wherein the p-tau level is a plasma p-tau¹⁸¹ or plasma p-tau²¹⁷ level.

14. The method of any one of embodiments 1-13, wherein the human subject has been diagnosed with mild cognitive impairment due to Alzheimer's disease.

15. The method of any one of embodiments 1-13, wherein the subject has been diagnosed with mild cognitive impairment due to Alzheimer's disease-intermediate likelihood by a CDR global score of 0.5 and a Memory Box score of 0.5 or greater before treatment.

16. The method of any one of embodiments 1-13, wherein the subject has been diagnosed with mild cognitive impairment due to Alzheimer's disease-intermediate likelihood by a history of subjective memory decline with gradual onset and slow progression over the last 1 year before treatment, e.g., as corroborated by an caregiver.

17. The method of any one of embodiments 1-13, wherein the subject has been diagnosed with mild Alzheimer's disease dementia by the NIA-AA core clinical criteria for probable Alzheimer's disease dementia.

18. The method of any one of embodiments 1-13, wherein the subject has been diagnosed with mild Alzheimer's disease dementia by a CDR score of 0.5 to 1.0 and a Memory Box score of 0.5 or greater before treatment.

19. The method of any one of embodiments 1-18, wherein the anti-beta-amyloid antibody is selected from: aducanumab, lecanemab, gantenerumab, donanemab, LY3002813, bapineuzumab, crenezumab, MEDI-1814, and solanezumab.

20. The method of any one of embodiments 1-18, wherein the anti-beta-amyloid antibody comprises:
a VHCDR1 with the amino acid sequence of SEQ ID NO:3, a VHCDR2 with the amino acid sequence of SEQ ID NO:4, and a VHCDR3 with the amino acid sequence of SEQ ID NO:5, and
a VLCDR1 with the amino acid sequence of SEQ ID NO:6, a VLCDR2 with the amino acid sequence of SEQ ID NO:7, and a VLCDR3 with the amino acid sequence of SEQ ID NO:8.

21. The method of any one of embodiments 1-18, wherein the anti-beta-amyloid antibody comprises a VH comprising the amino acid sequence of SEQ ID NO: 1 and a VL comprising the amino acid sequence of SEQ ID NO:2.

22. The method of any one of embodiments 1-18, wherein the anti-beta-amyloid antibody comprises a heavy chain comprising the amino acid sequence of SEQ ID NO:10 and a light chain comprising the amino acid sequence of SEQ ID NO:11.

23. The method of any one of embodiments 1-22, wherein the anti-beta-amyloid antibody is administered intravenously or subcutaneously.

24. The method of any one of embodiments 1-23, comprising administering a dose of the anti-beta-amyloid antibody in an amount of 3 mg antibody/kg of body weight of the human subject.

25 The method of any one of embodiments 1-24, comprising administering a dose of the anti-beta-amyloid antibody in an amount of 6 mg antibody/kg of body weight of the human subject.

26 The method of any one of embodiments 1-25, comprising administering a dose of the anti-beta-amyloid antibody in an amount of 10 mg antibody/kg of body weight of the human subject.

27. The method of any one of embodiments 1-26, comprising administering the anti-beta-amyloid antibody as a multiple dose regimen comprising multiple doses of 1 mg antibody/kg of body weight of the human patient are administered to the human patient at intervals of 4 weeks, multiple doses of 3 mg antibody/kg of body weight of the human patient are administered to the human patient at intervals of 4 weeks, multiple doses of 6 mg antibody/kg of body weight of the human patient are administered to the human patient at intervals of 4 weeks, and multiple doses of 10 mg antibody/kg of body weight of the human patient are administered to the human patient at intervals of 4 weeks.

28 The method of any one of embodiments 1-27, comprising:
(a) administering a dose of the anti-beta-amyloid antibody to the human subject in an amount of 1 mg antibody/kg of body weight of the human subject;
(b) 4 weeks after step (a), administering a dose of the antibody to the human subject in an amount of 1 mg antibody/kg of body weight of the human subject;
(c) 4 weeks after step (b), administering a dose of the antibody to the human subject in an amount of 3 mg antibody/kg of body weight of the human subject;
(d) 4 weeks after step (c), administering a dose of the antibody to the human subject in an amount of 3 mg antibody/kg of body weight of the human subject;
(e) 4 weeks after step (d), administering a dose of the antibody to the human subject in an amount of 6 mg antibody/kg of body weight of the human subject;
(f) 4 weeks after step (e), administering a dose of the antibody to the human subject in an amount of 6 mg antibody/kg of body weight of the human subject; and
(g) in consecutive intervals of 4 weeks after step (f), administering a dose of the antibody to the human subject in an amount of 10 mg antibody/kg of body weight of the human subject.

29. The method of any one of embodiments 1-27, comprising administering a dose of the antibody at a cumulative dose of at least 150 mg antibody/kg of body weight of the human subject.

30. The method of any one of embodiments 1-27, comprising administering a dose of the antibody at a cumulative dose of at least 200 mg antibody/kg of body weight of the human subject.

31. The method of any one of embodiments 1-27, comprising administering a dose of the antibody in an amount of 10 mg antibody/kg of body weight of the human subject every 4 weeks over at least 52 weeks.

32 The method of any one of embodiments 1-27, comprising administering a dose of the antibody in an amount of 6 mg antibody/kg of body weight of the human subject every 4 weeks over at least 112 weeks.

33 The method of any one of embodiments 1-27, comprising administering the antibody to the human subject in multiple doses and wherein the multiple doses comprise:
(a) at least two doses of 3 mg antibody/kg of body weight of the human subject every 4 weeks; and
(b) at least thirty doses of 6 mg antibody/kg of body weight of the human subject every 4 weeks.

34. The method of any one of embodiments 1-33, wherein the human subject is an ApoE carrier.

35. The method of any one of embodiments 1-33, wherein the human subject is an ApoE noncarrier.

36 The method of any one of embodiments 1-35, wherein the human subject is
(i) aged ≥65 years at baseline;
(ii) aged 65 to 74 years at baseline; or
(iii) aged ≥75 years.

37. The method of any one of embodiments 1-36, wherein the human subject does not develop an Amyloid Related Imaging Abnormality (ARIA) during the course of treatment that requires suspension of treatment.

38. The method of any one of embodiments 1-37, wherein the method is a method of treating Alzheimer's Disease (AD) in the human subject.

39 The method of any one of embodiments 1-38, wherein the method is a method of monitoring treatment response in the human subject.

40. The method of any one of embodiments 1-38, wherein the method is a method of determining a treatment plan for the human subject.

41. The method of any one of embodiments 1-38, wherein the method is a method of monitoring a cognitive state of the human subject.

42. A method, comprising
determining a p-tau level in plasma of a human subject who has received at least one dose of an anti-beta amyloid antibody, and
adjusting the amount of an anti-beta-amyloid antibody in a dose administered to the human subject based on the determined plasma p-tau level.

43. A method for selecting a patient for treatment with an anti-beta-amyloid antibody comprising determining a p-tau level in plasma of a human subject.

44. A method for determining a dose of an anti-beta-amyloid antibody to be administered to a human subject comprising determining a p-tau level in plasma of a human subject.

45. A method for selecting a patient receiving an anti-beta-amyloid antibody for cognitive support services comprising determining a p-tau level in plasma of a human subject.

46. A method, comprising:
determining a p-tau level in plasma of a human subject who has received at least one dose of an anti-beta-amyloid antibody.

47. The method of embodiment 46, comprising comparing the p-tau level in plasma of the human subject to a reference plasma p-tau level.

48. The method of embodiment 47, wherein the reference plasma p-tau level is a p-tau level in plasma of the human subject prior to receiving a dose of an anti-beta-amyloid antibody.

49. The method of embodiment 47, wherein the reference plasma p-tau level is an average p-tau level in plasma of a plurality of human subjects.

50 The method of embodiment 49, wherein the plurality of human subjects are a plurality of healthy human subjects with no evidence of AD.

51. The method of embodiment 49, wherein the plurality of human subjects are a plurality of human subjects with a known level of amyloid plaques.

52. The method of embodiment 49, wherein the plurality of human subjects are a plurality of human subjects with a known level of tau tangles.

53. The method of embodiment 49, wherein the plurality of human subjects are a plurality of human subjects with a predetermined level of cognition.

54 The method of any one of embodiments 47-53, wherein the human subject is classified as being susceptible to worsening cognition if the p-tau level in plasma of the human subject is higher than a reference plasma p-tau level.

55 The method of any one of embodiments 47-53, wherein the human subject is classified as being susceptible to increased numbers of amyloid plaques if the p-tau level in plasma of the human subject is higher than a reference plasma p-tau level.

56 The method of any one of embodiments 47-53, wherein the human subject is classified as being susceptible to increased numbers of tau tangles if the p-tau level in plasma of the human subject is higher than a reference plasma p-tau level.

57 The method of any one of embodiments 47-53, comprising administering another dose of the anti-beta-amyloid antibody to the human subject if the p-tau level in plasma of the human subject is higher than a reference plasma p-tau level.

58. The method of embodiment 57, wherein administering another dose of the anti-beta-amyloid antibody to the human subject comprises administering a dose of the anti-beta-amyloid antibody to the human subject that is equal to or greater than a prior dose of the anti-beta-amyloid antibody received by the human subject.

59. The method of any one of embodiments 1-58, wherein the plasma p-tau level is a plasma p-tau¹⁸¹ or p-tau²¹⁷ level.

60. A method comprising
determining a p-tau level in plasma of a human subject at baseline, and to a subject whose plasma p-tau level is above a reference level:
administering one or more doses of an anti-beta-amyloid antibody.

61. A composition for use in a method accordingly to any one of embodiments 1 to 60.

62. A kit comprising a composition of embodiment 61 and instructions for use.

63. Use of an anti-beta-amyloid antibody in the manufacture of a medicament for treating a disease in a human subject, wherein the treatment comprises
determining a p-tau level in plasma of the human subject, and
administering one or more doses of an anti-beta-amyloid antibody to the human subject, and wherein the disease comprises Alzheimer's Disease (AD).

64. The use of embodiment 63, wherein the p-tau level in plasma of a human subject is determined prior to the human subject receiving a dose of the anti-beta-amyloid antibody.

65. The use of embodiment 63 or 64, wherein the treatment further comprises determining a p-tau level in plasma of the human subject at a first and second time point.

66 The use of embodiment 65, wherein the first time point is prior to the human subject receiving a dose of the anti-beta-amyloid antibody.

67 The use of embodiment 65 or 66, wherein the second time point is after the human subject has received at least one dose of the anti-beta-amyloid antibody.

68. The use of any one of embodiments 65-67, wherein the second time point for determining the level of plasma p-tau is after at least 10 doses of anti-beta-amyloid antibody have been administered to the human subject.

69 The use of any one of embodiments 65-68, wherein the second time point for determining the level of plasma p-tau is after at least 14 doses of anti-beta-amyloid antibody have been administered to the human subject.

70 The use of any one of embodiments 65-69, wherein the second time point for determining the level of plasma p-tau is after at least 19 doses of anti-beta-amyloid antibody have been administered to the human subject.

71 The use of any one of embodiments 67-70, wherein the first time point occurs before the second time point, and, if the level of plasma p-tau at a first time point is greater than the level of plasma p-tau at the second time point, the treatment further comprises a second administration of the anti-beta-amyloid antibody to the human subject.

72 The use of embodiment 71, wherein the second administration of the anti-beta-amyloid antibody includes a dose of the anti-beta-amyloid antibody that is equal to or greater than the dose of the anti-beta-amyloid antibody administered to the human subject in the first administration.

73. The use of any one of embodiments 67-70, wherein the first time point occurs before the second time point, and, if the level of plasma p-tau at a first time point is less than the level of plasma p-tau at the second time point, the treatment further comprises a second administration of the anti-beta-amyloid antibody to the human subject.

74. The use of embodiment 73, wherein the second administration of the anti-beta-amyloid antibody includes a dose of the anti-beta-amyloid antibody that is equal to or less than the dose of the anti-beta-amyloid antibody administered to the human subject in the first administration.

75. The use of any one of embodiments 63-74, wherein the p-tau level is a plasma p-tau¹⁸¹ or plasma p-tau²¹⁷ level.

76. The use of any one of embodiments 63-75, wherein the human subject has been diagnosed with mild cognitive impairment due to Alzheimer's disease.

77 The use of any one of embodiments 63-75, wherein the subject has been diagnosed with mild cognitive impairment due to Alzheimer's disease-intermediate likelihood by a CDR global score of 0.5 and a Memory Box score of 0.5 or greater before treatment.

78 The use of any one of embodiments 63-75, wherein the subject has been diagnosed with mild cognitive impairment due to Alzheimer's disease-intermediate likelihood by a history of subjective memory decline with gradual onset and slow progression over the last 1 year before treatment, e.g., as corroborated by an caregiver.

79 The use of any one of embodiments 63-75, wherein the subject has been diagnosed with mild Alzheimer's disease dementia by the NIA-AA core clinical criteria for probable Alzheimer's disease dementia.

80. The use of any one of embodiments 63-75, wherein the subject has been diagnosed with mild Alzheimer's disease dementia by a CDR score of 0.5 to 1.0 and a Memory Box score of 0.5 or greater before treatment.

81 The use of any one of embodiments 63-80, wherein the anti-beta-amyloid antibody is selected from: aducanumab, lecanemab, gantenerumab, donanemab, LY3002813, bapineuzumab, crenezumab, MEDI-1814, and solanezumab.

82. The use of any one of embodiments 63-80, wherein the anti-beta-amyloid antibody comprises:
a VHCDR1 with the amino acid sequence of SEQ ID NO:3, a VHCDR2 with the amino acid sequence of SEQ ID NO:4, and a VHCDR3 with the amino acid sequence of SEQ ID NO:5, and
a VLCDR1 with the amino acid sequence of SEQ ID NO:6, a VLCDR2 with the amino acid sequence of SEQ ID NO:7, and a VLCDR3 with the amino acid sequence of SEQ ID NO:8.

83. The use of any one of embodiments 63-80, wherein the anti-beta-amyloid antibody comprises a VH comprising the amino acid sequence of SEQ ID NO: 1 and a VL comprising the amino acid sequence of SEQ ID NO:2.

84. The use of any one of embodiments 63-80, wherein the anti-beta-amyloid antibody comprises a heavy chain comprising the amino acid sequence of SEQ ID NO: 10 and a light chain comprising the amino acid sequence of SEQ ID NO:11.

85. The use of any one of embodiments 63-84, wherein the anti-beta-amyloid antibody is administered intravenously or subcutaneously.

86 The use of any one of embodiments 63-85, wherein the treatment comprises administering a dose of the anti-beta-amyloid antibody in an amount of 3 mg antibody/kg of body weight of the human subject.

87 The use of any one of embodiments 63-86, wherein the treatment comprises administering a dose of the anti-beta-amyloid antibody in an amount of 6 mg antibody/kg of body weight of the human subject.

88 The use of any one of embodiments 63-87, wherein the treatment comprises administering a dose of the anti-beta-amyloid antibody in an amount of 10 mg antibody/kg of body weight of the human subject.

89. The use of any one of embodiments 63-88, wherein the treatment comprises administering the anti-beta-amyloid antibody as a multiple dose regimen comprising multiple doses of 1 mg antibody/kg of body weight of the human patient are administered to the human patient at intervals of 4 weeks, multiple doses of 3 mg antibody/kg of body weight of the human patient are administered to the human patient at intervals of 4 weeks, multiple doses of 6 mg antibody/kg of body weight of the human patient are administered to the human patient at intervals of 4 weeks, and multiple doses of 10 mg antibody/kg of body weight of the human patient are administered to the human patient at intervals of 4 weeks.

90 The use of any one of embodiments 63-89, wherein the treatment comprises:
(a) administering a dose of the anti-beta-amyloid antibody to the human subject in an amount of 1 mg antibody/kg of body weight of the human subject;
(b) 4 weeks after step (a), administering a dose of the antibody to the human subject in an amount of 1 mg antibody/kg of body weight of the human subject;
(c) 4 weeks after step (b), administering a dose of the antibody to the human subject in an amount of 3 mg antibody/kg of body weight of the human subject;
(d) 4 weeks after step (c), administering a dose of the antibody to the human subject in an amount of 3 mg antibody/kg of body weight of the human subject;
(e) 4 weeks after step (d), administering a dose of the antibody to the human subject in an amount of 6 mg antibody/kg of body weight of the human subject;
(f) 4 weeks after step (e), administering a dose of the antibody to the human subject in an amount of 6 mg antibody/kg of body weight of the human subject; and
(g) in consecutive intervals of 4 weeks after step (f), administering a dose of the antibody to the human subject in an amount of 10 mg antibody/kg of body weight of the human subject.

91 The use of any one of embodiments 63-89, wherein the treatment comprises administering a dose of the antibody at a cumulative dose of at least 150 mg antibody/kg of body weight of the human subject.

92. The use of any one of embodiments 63-89, wherein the treatment comprises administering a dose of the antibody at a cumulative dose of at least 200 mg antibody/kg of body weight of the human subject.

93. The use of any one of embodiments 63-89, wherein the treatment comprises administering a dose of the antibody in an amount of 10 mg antibody/kg of body weight of the human subject every 4 weeks over at least 52 weeks.

94. The use of any one of embodiments 63-89, wherein the treatment comprises administering a dose of the antibody in an amount of 6 mg antibody/kg of body weight of the human subject every 4 weeks over at least 112 weeks.

95. The use of any one of embodiments 63-89, wherein the treatment comprises administering the antibody to the human subject in multiple doses and wherein the multiple doses comprise:
(a) at least two doses of 3 mg antibody/kg of body weight of the human subject every 4 weeks; and
(b) at least thirty doses of 6 mg antibody/kg of body weight of the human subject every 4 weeks.

96. The use of any one of embodiments 63-95, wherein the human subject is an ApoE carrier.

97. The use of any one of embodiments 63-95, wherein the human subject is an ApoE noncarrier.

98 The use of any one of embodiments 63-97, wherein the human subject is
(i) aged ≥65 years at baseline;
(ii) aged 65 to 74 years at baseline; or
(iii) aged ≥75 years.

99 The use of any one of embodiments 63-98, wherein the human subject does not develop an Amyloid Related Imaging Abnormality (ARIA) during the course of treatment that requires suspension of treatment.

100. The use of any one of embodiments 63-99, wherein the treatment comprises monitoring treatment response in the human subject.

101. The use of any one of embodiments 63-99, wherein the treatment comprises determining a treatment plan for the human subject.

102. The use of any one of embodiments 63-99, wherein the treatment comprises monitoring the cognitive state of the human subject.

103. A method, comprising
determining a p-tau level in plasma, wherein the plasma is an in vitro sample that had been obtained from a human subject who has received at least one dose of an anti-beta amyloid antibody, and
adjusting the amount of an anti-beta-amyloid antibody in a dose administered to the human subject based on the determined plasma p-tau level.

104. A method for selecting a patient for treatment with an anti-beta-amyloid antibody comprising determining a p-tau level in plasma, wherein the plasma is an in vitro sample that had been obtained from a human subject.

105. A method for determining a dose of an anti-beta-amyloid antibody to be administered to a human subject comprising determining a p-tau level in plasma, wherein the plasma is an in vitro sample that had been obtained from a human subject.

106. A method for selecting a patient receiving an anti-beta-amyloid antibody for cognitive support services comprising determining a p-tau level in plasma, wherein the plasma is an in vitro sample that had been obtained from a human subject.

107. A method, comprising:
determining a p-tau level in plasma, wherein the plasma is an in vitro sample that had been obtained from a human subject who has received at least one dose of an anti-beta-amyloid antibody.

108. The method of embodiment 107, comprising comparing the p-tau level the plasma to a reference plasma p-tau level.

109. The method of embodiment 108, wherein the reference plasma p-tau level is a p-tau level in plasma which was obtained from the human subject prior to receiving a dose of an anti-beta-amyloid antibody.

110. The method of embodiment 108, wherein the reference plasma p-tau level is an average p-tau level in plasma which was obtained from a plurality of human subjects.

111. The method of embodiment 110, wherein the plurality of human subjects are a plurality of healthy human subjects with no evidence of AD.

112. The method of embodiment 110, wherein the plurality of human subjects are a plurality of human subjects with a known level of amyloid plaques.

113. The method of embodiment 110, wherein the plurality of human subjects are a plurality of human subjects with a known level of tau tangles.

114. The method of embodiment 110, wherein the plurality of human subjects are a plurality of human subjects with a predetermined level of cognition.

115. The method of any one of embodiments 108-114, wherein the human subject is classified as being susceptible to worsening cognition if the p-tau level in the plasma is higher than a reference plasma p-tau level.

116. The method of any one of embodiments 108-114, wherein the human subject is classified as being susceptible to increased numbers of amyloid plaques if the p-tau level in the plasma is higher than a reference plasma p-tau level.

117. The method of any one of embodiments 108-114, wherein the human subject is classified as being susceptible to increased numbers of tau tangles if the p-tau level in the plasma is higher than a reference plasma p-tau level.

118. The method of any one of embodiments 108-114, comprising administering another dose of the anti-beta-amyloid antibody to the human subject if the p-tau level in the plasma is higher than a reference plasma p-tau level.

119. The method of embodiment 118, wherein administering another dose of the anti-beta-amyloid antibody to the human subject comprises administering a dose of the anti-beta-amyloid antibody to the human subject that is equal to or greater than a prior dose of the anti-beta-amyloid antibody received by the human subject.

120. The method of any one of embodiments 103-119, wherein the anti-beta-amyloid antibody is selected from: aducanumab, lecanemab, gantenerumab, donanemab, LY3002813, bapineuzumab, crenezumab, MEDI-1814, and solanezumab.

121. The use of any one of embodiments 63-102, wherein the plasma p-tau level is a plasma p-tau¹⁸¹ or p-tau²¹⁷ level.

122. The method of any one of embodiments 103-120, wherein the plasma p-tau level is a plasma p-tau¹⁸¹ or p-tau²¹⁷ level.

123. Use of the anti-beta-amyloid antibody in the manufacture of a medicament for treating a disease, wherein the treatment comprises
determining a p-tau level in plasma of a human subject at baseline, and to a subject whose plasma p-tau level is above a reference level:
administering one or more doses of an anti-beta-amyloid antibody.

124. A medicament formulated as a dosing regimen comprising an anti-beta-amyloid antibody for use in a method for treating Alzheimer's disease (AD) in a human subject, wherein the dosing regimen comprises multiple doses of an anti-beta-amyloid antibody and wherein a p-tau level in plasma of the human subject has been determined at a first time point and a second time point.

125. The medicament of embodiment 124, wherein the anti-beta-amyloid antibody is selected from: aducanumab, lecanemab, gantenerumab, donanemab, LY3002813, bapineuzumab, crenezumab, MEDI-1814, and solanezumab.

126. A medicament formulated as a dosing regimen comprising an anti-beta-amyloid antibody for use in a method, wherein the dosing regimen comprises multiple doses of an anti-beta-amyloid antibody, and wherein a p-tau level in plasma of the human subject has been determined at a first time point and a second time point, wherein the anti-beta-amyloid antibody comprises:
a VHCDR1 with the amino acid sequence of SEQ ID NO:3, a VHCDR2 with the amino acid sequence of SEQ ID NO:4, and a VHCDR3 with the amino acid sequence of SEQ ID NO:5, and
a VLCDR1 with the amino acid sequence of SEQ ID NO:6, a VLCDR2 with the amino acid sequence of SEQ ID NO:7, and a VLCDR3 with the amino acid sequence of SEQ ID NO:8.

127. The medicament of embodiment 126, wherein the method is:
(i) a method of treating Alzheimer's disease (AD),
(ii) a method of monitoring treatment response in a human subject with Alzheimer's disease (AD);
(iii) a method of determining a treatment plan for a human subject with Alzheimer's disease (AD);
(iv) a method of monitoring a cognitive state of a human subject with Alzheimer's disease (AD); and/or
(v) a method for selecting an Alzheimer's disease patient for treatment with the anti-beta-amyloid antibody.

128. The medicament of any one of embodiments 124-127, wherein the plasma p-tau level is a plasma p-tau¹⁸¹ or p-tau²¹⁷ level.

129. An anti-beta-amyloid antibody for use in a method for treating Alzheimer's disease (AD) in a human subject, wherein the treatment comprises
determining a plasma p-tau level in sample from the human subject, and
administering one or more doses of the anti-beta-amyloid antibody,
   wherein the anti-beta-amyloid antibody comprises:
a VHCDR1 with the amino acid sequence of SEQ ID NO:3, a VHCDR2 with the amino acid sequence of SEQ ID NO:4, and a VHCDR3 with the amino acid sequence of SEQ ID NO:5, and
a VLCDR1 with the amino acid sequence of SEQ ID NO:6, a VLCDR2 with the amino acid sequence of SEQ ID NO:7, and a VLCDR3 with the amino acid sequence of SEQ ID NO:8.

130. An anti-beta-amyloid antibody for use in a method for monitoring treatment response in a human subject with Alzheimer's disease (AD), wherein the method comprises
determining a plasma p-tau level in sample from the human subject, and
administering one or more doses of the anti-beta-amyloid antibody,
   wherein the anti-beta-amyloid antibody comprises:
a VHCDR1 with the amino acid sequence of SEQ ID NO:3, a VHCDR2 with the amino acid sequence of SEQ ID NO:4, and a VHCDR3 with the amino acid sequence of SEQ ID NO:5, and
a VLCDR1 with the amino acid sequence of SEQ ID NO:6, a VLCDR2 with the amino acid sequence of SEQ ID NO:7, and a VLCDR3 with the amino acid sequence of SEQ ID NO:8.

131. An anti-beta-amyloid antibody for use in a method for determining a treatment plan for a human subject with Alzheimer's disease (AD), wherein the method comprises
determining a plasma p-tau level in sample from the human subject, and
administering one or more doses of the anti-beta-amyloid antibody,
   wherein the anti-beta-amyloid antibody comprises:
a VHCDR1 with the amino acid sequence of SEQ ID NO:3, a VHCDR2 with the amino acid sequence of SEQ ID NO:4, and a VHCDR3 with the amino acid sequence of SEQ ID NO:5, and
a VLCDR1 with the amino acid sequence of SEQ ID NO:6, a VLCDR2 with the amino acid sequence of SEQ ID NO:7, and a VLCDR3 with the amino acid sequence of SEQ ID NO:8.

132. An anti-beta-amyloid antibody for use in a method for monitoring the cognitive state of a human subject with Alzheimer's disease (AD), wherein the method comprises
determining a plasma p-tau level in sample from the human subject, and
administering one or more doses of the anti-beta-amyloid antibody,
   wherein the anti-beta-amyloid antibody comprises:
a VHCDR1 with the amino acid sequence of SEQ ID NO:3, a VHCDR2 with the amino acid sequence of SEQ ID NO:4, and a VHCDR3 with the amino acid sequence of SEQ ID NO:5, and
a VLCDR1 with the amino acid sequence of SEQ ID NO:6, a VLCDR2 with the amino acid sequence of SEQ ID NO:7, and a VLCDR3 with the amino acid sequence of SEQ ID NO:8.

133. The anti-beta-amyloid antibody of embodiments 129-132, wherein the plasma p-tau level is a plasma p-tau¹⁸¹ or p-tau²¹⁷ level.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method comprising:
determining a p-tau level in plasma of a human subject, and
administering one or more doses of an anti-beta-amyloid antibody to the human subject, wherein the anti-beta-amyloid antibody comprises:
a VHCDR1 with the amino acid sequence of SEQ ID NO:3, a VHCDR2 with the amino acid sequence of SEQ ID NO:4, and a VHCDR3 with the amino acid sequence of SEQ ID NO:5, and
a VLCDR1 with the amino acid sequence of SEQ ID NO:6, a VLCDR2 with the amino acid sequence of SEQ ID NO:7, and a VLCDR3 with the amino acid sequence of SEQ ID NO:8.

2. The method of claim 1, wherein determining a p-tau level in plasma of the human subject is at a first time point and further comprises determining a p-tau level in plasma of the human subject at a second time point.

3. The method of claim 1 or 2, wherein the first time point is prior to the human subject receiving a dose of the anti-beta-amyloid antibody.

4. The method of any one of claims 1-3, wherein the second time point is after the human subject has received at least one dose of the anti-beta-amyloid antibody.

5. The method of any one of claims 1-3, wherein the second time point for determining the level of plasma p-tau is:
(i) after at least 10 doses of anti-beta-amyloid antibody have been administered to the human subject.
(ii) after at least 14 doses of anti-beta-amyloid antibody have been administered to the human subject, and/or
(iii) after at least 19 doses of anti-beta-amyloid antibody have been administered to the human subject.

6. The method of any one of claims 1-5, wherein the first time point occurs before the second time point, and, if the level of plasma p-tau at a first time point is greater than the level of plasma p-tau at the second time point, the method further comprises a second administration of the anti-beta-amyloid antibody to the human subject.

7. The method of claim 6, wherein the second administration of the anti-beta-amyloid antibody includes a dose of the anti-beta-amyloid antibody that is equal to or greater than the dose of the anti-beta-amyloid antibody administered to the human subject in the first administration.

8. The method of any one of claims 1-5, wherein the first time point occurs before the second time point, and, if the level of plasma p-tau at a first time point is less than the level of plasma p-tau at the second time point, the method further comprises a second administration of the anti-beta-amyloid antibody to the human subject.

9. The method of claim 8, wherein the second administration of the anti-beta-amyloid antibody includes a dose of the anti-beta-amyloid antibody that is equal to or less than the dose of the anti-beta-amyloid antibody administered to the human subject in the first administration.

10. A method comprising
determining a baseline p-tau level in plasma of a human subject, and to a human subject whose plasma p-tau level is above a reference level:
administering one or more doses of an anti-beta-amyloid antibody,
wherein the anti-beta-amyloid antibody comprises:
a VHCDR1 with the amino acid sequence of SEQ ID NO:3, a VHCDR2 with the amino acid sequence of SEQ ID NO:4, and a VHCDR3 with the amino acid sequence of SEQ ID NO:5, and
a VLCDR1 with the amino acid sequence of SEQ ID NO:6, a VLCDR2 with the amino acid sequence of SEQ ID NO:7, and a VLCDR3 with the amino acid sequence of SEQ ID NO:8.

11. The method of any one of claims 1-10, wherein the subject has been diagnosed with mild cognitive impairment due to Alzheimer's disease.

12. The method of claim 11, wherein the subject has been diagnosed with mild cognitive impairment due to Alzheimer's disease-intermediate likelihood by a CDR global score of 0.5 and a Memory Box score of 0.5 or greater before treatment.

13. The method claim 11 or 12, wherein the subject has been diagnosed with mild cognitive impairment due to Alzheimer's disease-intermediate likelihood by a history of subjective memory decline with gradual onset and slow progression over the last 1 year before treatment, e.g., as corroborated by an caregiver.

14. The method claim 11 or 12, wherein the subject has been diagnosed with mild Alzheimer's disease dementia by the NIA-AA core clinical criteria for probable Alzheimer's disease dementia.

15. The method claim 11 or 12, wherein the subject has been diagnosed with mild Alzheimer's disease dementia by a CDR score of 0.5 to 1.0 and a Memory Box score of 0.5 or greater before treatment.

16. The method of any one of claims 1-15, wherein the anti-beta-amyloid antibody is administered intravenously or subcutaneously.

17. The method of any one of claims 1-16, comprising administering a dose of the anti-beta-amyloid antibody in an amount of:
(i) 3 mg antibody/kg of body weight of the human subject;
(ii) 6 mg antibody/kg of body weight of the human subject; and/or
(iii) 10 mg antibody/kg of body weight of the human subject.

18. The method of any one of claims 1-17, comprising administering the anti-beta-amyloid antibody as a multiple dose regimen comprising multiple doses of 3 mg antibody/kg of body weight of the human patient are administered to the human patient at intervals of 4 weeks, multiple doses of 6 mg antibody/kg of body weight of the human patient are administered to the human patient at intervals of 4 weeks, and multiple doses of 10 mg antibody/kg of body weight of the human patient are administered to the human patient at intervals of 4 weeks.

19. The method of any one of claims 1-18, comprising:
(a) administering a dose of the anti-beta-amyloid antibody to the human subject in an amount of 1 mg antibody/kg of body weight of the human subject;
(b) 4 weeks after step (a), administering a dose of the antibody to the human subject in an amount of 1 mg antibody/kg of body weight of the human subject;
(c) 4 weeks after step (b), administering a dose of the antibody to the human subject in an amount of 3 mg antibody/kg of body weight of the human subject;
(d) 4 weeks after step (c), administering a dose of the antibody to the human subject in an amount of 3 mg antibody/kg of body weight of the human subject;
(e) 4 weeks after step (d), administering a dose of the antibody to the human subject in an amount of 6 mg antibody/kg of body weight of the human subject;
(f) 4 weeks after step (e), administering a dose of the antibody to the human subject in an amount of 6 mg antibody/kg of body weight of the human subject; and
(g) in consecutive intervals of 4 weeks after step (f), administering a dose of the antibody to the human subject in an amount of 10 mg antibody/kg of body weight of the human subject.

20. The method of any one of claims 1-19, comprising administering a dose of the antibody in an amount of 10 mg antibody/kg of body weight of the human subject every 4 weeks over at least 52 weeks.

21. The method of any one of claims 1-20, comprising administering a dose of the antibody in an amount of 6 mg antibody/kg of body weight of the human subject every 4 weeks over at least 112 weeks.

22. The method of any one of claims 1-18, comprising administering the antibody to the human subject in multiple doses and wherein the multiple doses comprise:
(a) at least two doses of 3 mg antibody/kg of body weight of the human subject every 4 weeks; and
(b) at least thirty doses of 6 mg antibody/kg of body weight of the human subject every 4 weeks.

23. The method of any one of claims 1-22, wherein the human subject does not develop an Amyloid Related Imaging Abnormality (ARIA) during the course of treatment that requires suspension of treatment.

24. The method of any one of claims 1-23, wherein the method is a method of treating Alzheimer's Disease (AD) in the human subject.

25. The method of any one of claims 1-23, wherein the method is a method of monitoring treatment response in the human subject.

26. The method of any one of claims 1-23, wherein the method is a method of determining a treatment plan for the human subject.

27. The method of any one of claims 1-23, wherein the method is a method of monitoring a cognitive state of the human subject.

28. The method of any one of claims 1-23, wherein the method is a method for selecting a patient for treatment with the anti-beta-amyloid antibody.

29. A method, comprising:
determining a p-tau level in plasma of a human subject who has received at least one dose of an anti-beta-amyloid antibody,
comparing the p-tau level in plasma of the human subject to a reference plasma p-tau level,
wherein the anti-beta-amyloid antibody comprises:
a VHCDR1 with the amino acid sequence of SEQ ID NO:3, a VHCDR2 with the amino acid sequence of SEQ ID NO:4, and a VHCDR3 with the amino acid sequence of SEQ ID NO:5, and
a VLCDR1 with the amino acid sequence of SEQ ID NO:6, a VLCDR2 with the amino acid sequence of SEQ ID NO:7, and a VLCDR3 with the amino acid sequence of SEQ ID NO:8.

30. The method of claim 29, wherein the reference plasma p-tau level is a p-tau level in plasma of the human subject prior to receiving a dose of an anti-beta-amyloid antibody.

31. The method of claim 29, wherein the reference plasma p-tau level is an average p-tau level in plasma of a plurality of human subjects.

32. The method of claim 31, wherein the plurality of human subjects are
(i) a plurality of healthy human subjects with no evidence of AD;
(ii) a plurality of human subjects with a known level of amyloid plaques;
(iii) a plurality of human subjects with a known level of tau tangles; or
(iv) a plurality of human subjects with a predetermined level of cognition.

33. The method of any one of claims 29-32, wherein the human subject is classified as being susceptible to worsening cognition if the p-tau level in plasma of the human subject is higher than a reference plasma p-tau level.

34. The method of any one of claims 29-32, wherein the human subject is classified as being susceptible to increased numbers of amyloid plaques if the p-tau level in plasma of the human subject is higher than a reference plasma p-tau level.

35. The method of any one of claims 29-32, wherein the human subject is classified as being susceptible to increased numbers of tau tangles if the p-tau level in plasma of the human subject is higher than a reference plasma p-tau level.

36. The method of any one of claims 29-32, comprising administering another dose of the anti-beta-amyloid antibody to the human subject if the p-tau level in plasma of the human subject is higher than a reference plasma p-tau level.

37. The method of claim 36, wherein administering another dose of the anti-beta-amyloid antibody to the human subject comprises administering a dose of the anti-beta-amyloid antibody to the human subject that is equal to or greater than a prior dose of the anti-beta-amyloid antibody received by the human subject.

38. The method of any one of claims 1-37, wherein the anti-beta-amyloid antibody comprises a VH comprising the amino acid sequence of SEQ ID NO: 1 and a VL comprising the amino acid sequence of SEQ ID NO:2.

39. The method of any one of claims 1-38, wherein the anti-beta-amyloid antibody comprises comprises a heavy chain comprising the amino acid sequence of SEQ ID NO: 10 and a light chain comprising the amino acid sequence of SEQ ID NO:11.

40. The method of any one of claims 1-39, wherein the human subject is an ApoE carrier.

41. The method of any one of claims 1-39, wherein the human subject is an ApoE noncarrier.

42. The method of any one of claims 1-41, wherein the human subject is
(i) aged ≥65 years at baseline;
(ii) aged 65 to 74 years at baseline; or
(iii) aged ≥75 years.

43. The method of any one of claims 1-42, wherein the plasma p-tau level is a plasma p-tau¹⁸¹ or p-tau²¹⁷ level.

44. A composition for use in a method accordingly to any one of claims 1 to 43.

45. A kit comprising a composition of claim 44 and instructions for use.

46. Use of an anti-beta-amyloid antibody in the manufacture of a medicament for treating Alzheimer's Disease (AD) in a human subject, wherein the treatment comprises
determining a p-tau level in plasma of the human subject, and
administering one or more doses of an anti-beta-amyloid antibody to the human subject, and wherein the anti-beta-amyloid antibody comprises:
a VHCDR1 with the amino acid sequence of SEQ ID NO:3, a VHCDR2 with the amino acid sequence of SEQ ID NO:4, and a VHCDR3 with the amino acid sequence of SEQ ID NO:5, and
a VLCDR1 with the amino acid sequence of SEQ ID NO:6, a VLCDR2 with the amino acid sequence of SEQ ID NO:7, and a VLCDR3 with the amino acid sequence of SEQ ID NO:8.

47. Use of an anti-beta-amyloid antibody in the manufacture of a medicament for treating Alzheimer's Disease (AD) in a human subject, wherein the treatment comprises:
determining a p-tau level in plasma of a human subject, and to a human subject whose plasma p-tau level is above a reference level:
administering one or more doses of an anti-beta-amyloid antibody,
wherein the anti-beta-amyloid antibody comprises:
a VHCDR1 with the amino acid sequence of SEQ ID NO:3, a VHCDR2 with the amino acid sequence of SEQ ID NO:4, and a VHCDR3 with the amino acid sequence of SEQ ID NO:5, and
a VLCDR1 with the amino acid sequence of SEQ ID NO:6, a VLCDR2 with the amino acid sequence of SEQ ID NO:7, and a VLCDR3 with the amino acid sequence of SEQ ID NO:8.

48. The use of claim 46 or 47, wherein the subject has been diagnosed with mild cognitive impairment due to Alzheimer's disease.

49. The use of claim 48, wherein the mild cognitive impairment due to Alzheimer's disease is diagnosed by
(i) a CDR global score of 0.5 and a Memory Box score of 0.5 or greater before treatment;
(ii) a history of subjective memory decline with gradual onset and slow progression over the last 1 year before treatment, e.g., as corroborated by an caregiver;
(iii) by the NIA-AA core clinical criteria for probable Alzheimer's disease dementia; and/or
(iv) a CDR score of 0.5 to 1.0 and a Memory Box score of 0.5 or greater before treatment.

50. The use of any one of claims 46-49, wherein the anti-beta-amyloid antibody is administered intravenously or subcutaneously.

51. The use of any one of claims 46-50, wherein the anti-beta-amyloid antibody comprises a VH comprising the amino acid sequence of SEQ ID NO: 1 and a VL comprising the amino acid sequence of SEQ ID NO:2.

52. The use of any one of claims 46-51, wherein the anti-beta-amyloid antibody comprises comprises a heavy chain comprising the amino acid sequence of SEQ ID NO:10 and a light chain comprising the amino acid sequence of SEQ ID NO:11.

53. The use of any one of claims 46-52, wherein the human subject is an ApoE carrier.

54. The use of any one of claims 46-52, wherein the human subject is an ApoE noncarrier.

55. The use of any one of claims 46-54, wherein the human subject is
(i) aged ≥65 years at baseline;
(ii) aged 65 to 74 years at baseline; or
(iii) aged ≥75 years.

56. The use of any one of claims 46-55, wherein the plasma p-tau level is a plasma p-tau¹⁸¹ or p-tau²¹⁷ level.

57. An anti-beta-amyloid antibody for use in a method for treating Alzheimer's disease (AD) in a human subject, wherein the treatment comprises
determining a plasma p-tau level in sample from the human subject, and
administering one or more doses of the anti-beta-amyloid antibody,
wherein the anti-beta-amyloid antibody comprises:
a VHCDR1 with the amino acid sequence of SEQ ID NO:3, a VHCDR2 with the amino acid sequence of SEQ ID NO:4, and a VHCDR3 with the amino acid sequence of SEQ ID NO:5, and
a VLCDR1 with the amino acid sequence of SEQ ID NO:6, a VLCDR2 with the amino acid sequence of SEQ ID NO:7, and a VLCDR3 with the amino acid sequence of SEQ ID NO:8.

58. An anti-beta-amyloid antibody for use in a method for monitoring treatment response in a human subject with Alzheimer's disease (AD), wherein the method comprises
determining a plasma p-tau level in sample from the human subject, and
administering one or more doses of the anti-beta-amyloid antibody,
wherein the anti-beta-amyloid antibody comprises:
a VHCDR1 with the amino acid sequence of SEQ ID NO:3, a VHCDR2 with the amino acid sequence of SEQ ID NO:4, and a VHCDR3 with the amino acid sequence of SEQ ID NO:5, and
a VLCDR1 with the amino acid sequence of SEQ ID NO:6, a VLCDR2 with the amino acid sequence of SEQ ID NO:7, and a VLCDR3 with the amino acid sequence of SEQ ID NO:8.

59. An anti-beta-amyloid antibody for use in a method for determining a treatment plan for a human subject with Alzheimer's disease (AD), wherein the method comprises
determining a plasma p-tau level in sample from the human subject, and
administering one or more doses of the anti-beta-amyloid antibody,
wherein the anti-beta-amyloid antibody comprises:
a VHCDR1 with the amino acid sequence of SEQ ID NO:3, a VHCDR2 with the amino acid sequence of SEQ ID NO:4, and a VHCDR3 with the amino acid sequence of SEQ ID NO:5, and
a VLCDR1 with the amino acid sequence of SEQ ID NO:6, a VLCDR2 with the amino acid sequence of SEQ ID NO:7, and a VLCDR3 with the amino acid sequence of SEQ ID NO:8.

60. An anti-beta-amyloid antibody for use in a method for monitoring the cognitive state of a human subject with Alzheimer's disease (AD), wherein the method comprises
determining a plasma p-tau level in sample from the human subject, and
administering one or more doses of the anti-beta-amyloid antibody,
wherein the anti-beta-amyloid antibody comprises:
a VHCDR1 with the amino acid sequence of SEQ ID NO:3, a VHCDR2 with the amino acid sequence of SEQ ID NO:4, and a VHCDR3 with the amino acid sequence of SEQ ID NO:5, and
a VLCDR1 with the amino acid sequence of SEQ ID NO:6, a VLCDR2 with the amino acid sequence of SEQ ID NO:7, and a VLCDR3 with the amino acid sequence of SEQ ID NO:8.

61. The anti-beta-amyloid antibody of any one of claims 57-60, wherein the plasma p-tau level is determined at a first and second time point.

62. The anti-beta-amyloid antibody of any one of claims 57-61, wherein the plasma p-tau level is a plasma p-tau¹⁸¹ or p-tau²¹⁷ level.

63. A medicament formulated as a dosing regimen comprising an anti-beta-amyloid antibody for use in a method, wherein the dosing regimen comprises multiple doses of an anti-beta-amyloid antibody, and wherein a p-tau level in plasma of the human subject has been determined at a first time point and a second time point,
wherein the anti-beta-amyloid antibody comprises:
a VHCDR1 with the amino acid sequence of SEQ ID NO:3, a VHCDR2 with the amino acid sequence of SEQ ID NO:4, and a VHCDR3 with the amino acid sequence of SEQ ID NO:5, and
a VLCDR1 with the amino acid sequence of SEQ ID NO:6, a VLCDR2 with the amino acid sequence of SEQ ID NO:7, and a VLCDR3 with the amino acid sequence of SEQ ID NO:8.

64. The medicament of claim 63, wherein the method is:
(i) a method of treating Alzheimer's disease (AD),
(ii) a method of monitoring treatment response in a human subject with Alzheimer's disease (AD);
(iii) a method of determining a treatment plan for a human subject with Alzheimer's disease (AD);
(iv) a method of monitoring a cognitive state of a human subject with Alzheimer's disease (AD); and/or
(v) a method for selecting an Alzheimer's disease patient for treatment with the anti-beta-amyloid antibody.

65. The medicament of claim 63 or 64, wherein the plasma p-tau level is a plasma p-tau¹⁸¹ or p-tau²¹⁷ level.
